SEQUENCE LISTING (8 sequences)

SEQ ID NO: 1 - protein, 5 aa, synthetic construct
NYYMY

SEQ ID NO: 2 - protein, 16 aa, synthetic construct
GINPSNGGTNFNEKFK

SEQ ID NO: 3 - protein, 11 aa, synthetic construct
RDYRFDMGFDY

SEQ ID NO: 4 - protein, 15 aa, synthetic construct
RASKGVSTSGYSYLH

SEQ ID NO: 5 - protein, 7 aa, synthetic construct
LASYLES

SEQ ID NO: 6 - protein, 9 aa, synthetic construct
QHSRDLPLT

SEQ ID NO: 7 - protein, 446 aa, synthetic construct
VQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 217 aa, synthetic construct
IVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0215086 A1
(43) Pub. Date: Jul. 3, 2025

(54) ANTI-PD-1 ANTIBODY FORMULATIONS

(71) Applicant: Amgen Inc., Thousand Oaks, CA (US)

(72) Inventors: Shantanu Sule, Thousand Oaks, CA (US); Li Gu, Thousand Oaks, CA (US); Kelsey O'Donnell, Thousand Oaks, CA (US); Monica M. Goss, Thousand Oaks, CA (US)

(73) Assignee: Amgen Inc., Thousand Oaks, CA (US)

(21) Appl. No.: 18/878,828

(22) PCT Filed: Jun. 30, 2023
(86) PCT No.: PCT/US2023/069486

§ 371 Date: Dec. 24, 2024

Related U.S. Application Data
(60) Provisional application No. 63/358,033, filed on Jul. 1, 2022.

(51) Int. Cl. C07K 16/28; A61K 39/00; A61K 45/06; A61K 47/10; A61K 47/18; A61K 47/26

(52) U.S. Cl. CPC C07K 16/2818; A61K 45/06; A61K 47/10; A61K 47/183; A61K 47/26; A61K 2039/54; A61K 2039/545

(57) ABSTRACT

In some aspects, the present disclosure describes stable formulations comprising pembrolizumab, methods of making stable pharmaceutical formulations thereof, and methods of using stable pharmaceutical formulations thereof.

### CROSS REFERENCE TO RELATED APPLICATION

This application claims the benefit under 35 U.S.C. § 119(e) of U.S. Provisional Application Ser. No. 63/358,033, entitled “ANTI-PD-1 ANTIBODY FORMULATIONS,” filed on Jul. 1, 2022, which is hereby incorporated by reference in its entirety.

### REFERENCE TO AN ELECTRONIC SEQUENCE LISTING

The contents of the electronic sequence listing (A117570000WO00-SEQ-AZW.xml; Size: 8,546 bytes; and Date of Creation: Jun. 27, 2023) is herein incorporated by reference in its entirety.

### FIELD OF THE INVENTION

The present disclosure relates to stable formulations comprising pembrolizumab or a biosimilar thereof, and methods of making and using such formulations.

### BACKGROUND

PD-1 is an inhibitory immune checkpoint present on the surface of T-cells. Activation of the PD-1 signaling pathway through the interaction of PD-1 with a ligand (e.g., PD-L1 or PD-L2) leads to the suppression of immune cell reaction. This mechanism is observed in cancers and viral infections. For example, a mechanism used by cancer cells to evade immune cells is the secretion of ligands targeted to inhibitory immune checkpoints (e.g., PD-1) on the surface of T-cells. Anti-PD-1 antibodies bind to PD-1 and block its interaction with its ligands, thereby preventing the inhibition of T-cell response against cancer cells. One such antibody is pembrolizumab, a recombinant monoclonal antibody specific for human PD-1.

### SUMMARY

The present disclosure is directed to stable pharmaceutical formulations comprising pembrolizumab, to methods of making stable pharmaceutical formulations comprising pembrolizumab, to uses of formulations as disclosed herein, and to methods of treating a disease comprising administering a formulation as disclosed herein to a subject.

Surprisingly, some embodiments of the stable formulations disclosed herein have self-buffering capabilities and/or are stable at 2-8 degrees Celsius, pH 5.3-5.8, such as 5.5, for extended periods of times in the absence of one or more strong buffering agents, such as histidine, and/or one or more disaccharides. Commercially available pembrolizumab formulations even in the presence of one or more strong buffering agents (e.g., histidine) and disaccharides (i.e., sucrose) are not recommended to be stored for more than 6 hours at room temperature or for more than 96 hours at 2-8 degrees Celsius from the time of dilution.

In some embodiments, disclosed formulations include amino acids not previously known to be effective stabilizing reagents in monoclonal antibody formulations, let alone PD-1 inhibitor specific, liquid formulations at pH 5.2 to 5.8. For example, it is believed that neither L-threonine nor asparagine were previously known to be effective stabilizing agents in monoclonal antibody formulations let alone PD-1 specific formulations prior to the present studies. In some embodiments, the disclosure provides a pharmaceutical formulation comprising: (i) about 25 mg/mL to 200 mg/mL, such as about 25 mg/mL or about 50 mg/mL of an anti-PD-1 antibody, such as pembrolizumab, (ii) a surfactant, and (iii) a stabilizing agent, such as one or more stabilizing agents, such as two, three, four, or more stabilizing agents. In some embodiments, the disclosure provides a pharmaceutical formulation comprising: (i) about 25 mg/mL to 200 mg/mL, such as 25 mg/mL or 50 mg/mL of an anti-PD-1 antibody, such as pembrolizumab, (ii) a surfactant, and (iii) a stabilizing agent, such as one or more stabilizing agents, such as two, three, four, or more stabilizing agents and no buffer, with a pH between 5.2-5-8, such as 5.5.

In some embodiments, the disclosure provides a pharmaceutical formulation comprising: (i) about 25 mg/mL or 50 mg/mL pembrolizumab, (ii) a surfactant, and (iii) two stabilizing agents and wherein the two stabilizing agents are not disaccharide stabilizing agents and do not have strong buffering capabilities at pH 5.5.

In some embodiments, the anti-PD-1 antibody is pembrolizumab. It is to be understood that the term pembrolizumab includes a pembrolizumab biosimilar.

In some embodiments, the surfactant is a non-ionic surfactant. In some embodiments, the surfactant is polysorbate, such as polysorbate 80 (PS80), including about 0.01% to 0.1% w/v, or 0.02% w/v PS80.

In some embodiments, one or more stabilizing agents is an amino acid. In some embodiments, the amino acid is glutamic acid, such as L-glutamic acid, proline, valine, L-threonine, asparagine or any combination thereof. In some embodiments, the pembrolizumab formulation includes two amino acids, L-glutamic acid and valine and is at a pH between about 5.2 and 5.8, such as about 5.2, about 5.3, about 5.4, about 5.5, about 5.6, about 5.7 or about pH 5.8, including pH 5.2, 5.3, 5.4, 5.5, 5.6, 5.7 or 5.8. In some embodiments, the pembrolizumab formulation includes two amino acids, L-glutamic acid and asparagine and is at a pH between about 5.2 and 5.8, such as about 5.2, about 5.3, about 5.4, about 5.5, about 5.6, about 5.7 or about pH 5.8, including pH 5.2 and 5.8, such as pH 5.2, 5.3, 5.4, 5.5, 5.6, 5.7 or 5.8. In some embodiments, the pembrolizumab formulation includes two amino acids, L-glutamic acid and L-threonine and is at a pH between about 5.2 and 5.8, such as about 5.2, about 5.3, about 5.4, about 5.5, about 5.6, about 5.7 or about pH 5.8, including between 5.2 and 5.8, such as pH 5.2, 5.3, 5.4, 5.5, 5.6, 5.7 or 5.8. In some embodiments, the amino acid stabilizing agent is valine. In some embodiments, the amino acid stabilizing agent is L-threonine. In some embodiments, the stabilizing agent is a high concentration of L-threonine, such as greater than 100 mM L-threonine, including between 100 mM and 300 mM, 100 mM and 271 mM, 150 mM and 300 mM, 150 mM and 271 mM, 200 mM and 300 mM, 200 mM and 271 mM, 225 mM and 275 mM, 225 mM and 271 mM, 250 mM and 275 mM, 250 mM and 271 mM, including 100 mM, 125 mM, 150 mM, 175 mM, 200 mM, 225 mM, 250 mM, 251 mM, 252 mM, 253 mM, 254 mM, 255 mM, 256 mM, 257 mM, 258 mM, 259 mM, 260 mM, 261 mM, 262 mM, 263 mM, 264 mM, 265 mM, 266 mM, 267 mM, 268 mM, 269 mM, 270 mM, 271 mM, 272 mM, 273 mM, 274 mM, 275 mM, 276 mM, 277 mM, 278 mM, 279 mM, 280 mM, 281 mM, 282 mM, 283 mM, 284 mM, 285 mM, 286 mM, 289 mM, 290 mM, 291 mM, 292 mM, 293 mM, 294 mM, 295 mM, 296 mM, 297 mM, 298 mM, 299 mM or 300 mM. In some embodiments, the amino acid stabilizing agent is proline. In some embodiments, the stabilizing agent is a polyol. In some embodiments, the polyol stabilizing agent is glycerol. In some embodiments, the polyol stabilizing agent is sorbitol. In some embodiments, the polyol stabilizing agent is mannitol. In some embodiments, the stabilizing agent is not a disaccharide. In other embodiments, the stabilizing agent is a disaccharide. In some embodiments, the disaccharide stabilizing agent is trehalose. In some embodiments, the disaccharide stabilizing agent is sucrose. In some embodiments, the formulation comprises about 10% w/v trehalose. In some embodiments, the formulation does not include sucrose, sorbitol, glycerol and/or trehalose. In some embodiments, the formulation does not include histidine, sucrose, sorbitol, glycerol, trehalose or any combination thereof. In some embodiments, the formulation does not include histidine, trehalose, sucrose, acetic acid, glycerol or any combination thereof.

In some embodiments, the formulation comprises about 10 mM L-glutamic acid and about 2.5% w/v glycerol. In some embodiments, the formulation comprises about 10 mM L-glutamic acid and about 271 mM valine. In some embodiments, the formulation comprises about 10 mM L-glutamic acid, about 65 mM valine, and about 2% w/v glycerol. In some embodiments, the formulation comprises about 10 mM L-glutamic acid, about 5 mM valine, and about 2.5% w/v glycerol. In some embodiments, the formulation comprises about 10 mM L-glutamic acid and about 271 mM L-threonine. In some embodiments, the formulation comprises about 10 mM L-glutamic acid and about 250 mM L-threonine. In some embodiments, the formulation comprises about 10 mM L-glutamic acid, about 30 mM L-threonine, and about 2% w/v glycerol. In some embodiments, the formulation comprises about 10 mM L-glutamic acid, about 10 mM L-threonine, and about 2.5% w/v glycerol. In some embodiments, the formulation comprises about 10 mM L-glutamic acid, about 100 mM proline, and about 1.6% w/v glycerol. In some embodiments, the formulation comprises about 100 mM proline and about 1.6% w/v glycerol. In some embodiments, the formulation comprises about 10 mM L-glutamic acid and about 10% w/v trehalose. In some embodiments, the formulation comprises about 10 mM L-glutamic acid, about 1% w/v trehalose, and about 2% w/v glycerol. In some embodiments, the formulation comprises about 10 mM L-glutamic acid, about 1% w/v sorbitol, and about 2% w/v glycerol. In some embodiments, the formulation comprises about 10 mM L-glutamic acid, about 1% w/v mannitol, and about 2% w/v glycerol. In some embodiments, the formulation comprises about 2 mM acetic acid, about 10 mM L-glutamic acid, and about 2.5% w/v glycerol. In some embodiments, the formulation comprises about 10 mM lactic acid and about 2.5% w/v glycerol. In some embodiments, the formulation comprises about 10 mM lactic acid and about 271 mM L-threonine.

In some embodiments, the formulation includes about 25 mg/mL to 50 mg/mL of pembrolizumab, about 10 mM L-glutamic acid, about 271 mM valine, and about 0.02% w/v PS80 at pH 5.5.

In some embodiments, the formulation includes about 25 mg/mL to 50 mg/mL of pembrolizumab, about 10 mM L-glutamic acid, about 271 mM -L-threonine, and about 0.02% w/v PS80 at pH 5.5.

In some embodiments, the formulation includes about 25 mg/mL to 50 mg/mL of pembrolizumab, about 10 mM L-glutamic acid, about 250 mM -L-threonine, and about 0.02% w/v PS80 at pH 5.5.

In some embodiments, the disclosure provides a formulation comprising about 25 mg/mL or about 50 mg/mL of an anti-PD-1 antibody, wherein the formulation does not comprise a buffer and/or a disaccharide stabilizing agent.

In some embodiments, the formulation is self-buffering and/or does not include a buffering agent or a buffering agent that is at a pH at which it does not have strong buffering capabilities.

In other embodiments, a formulation includes one or more buffering reagents, such as lactic acid or acetic acid. In some embodiments, a disclosed formulation does not include a histidine buffer.

In some embodiments, the disclosure provides a method of treatment comprising administering a stable pharmaceutical formulation as described herein to a subject having or at risk of developing a disease or condition. In some embodiments, the method further comprises administering to the subject a second therapeutic composition. In some embodiments, the disease or condition is selected from the group consisting of: cancer, melanoma, renal cell carcinoma (RCC), non-small-cell lung cancer, bladder cancer, head and neck squamous cell cancer (HNSCC), classical Hodgkin lymphoma (cHL), primary mediastinal large B-cell lymphoma (PMBCL), urothelial carcinoma, microsatellite instability-high or mismatch repair deficient cancer, microsatellite instability-high or mismatch repair deficient colorectal cancer, gastric cancer, esophageal cancer, cervical cancer, hepatocellular carcinoma (HCC), Merkel cell carcinoma, endometrial carcinoma, tumor mutational burden-high (TMB-H) cancer, cutaneous squamous cell carcinoma (cSCC), triple-negative breast cancer (TNBC), anaplastic thyroid cancer, and infectious disease. In some embodiments, the subject is a mammal. In some embodiments, the mammal is a human.

Each of the limitations of the invention can encompass various embodiments of the invention. It is, therefore, anticipated that each of the limitations of the invention involving any one element or combinations of elements can be included in each aspect of the invention. This disclosure is not limited in its application to the details of construction and the arrangement of components set forth in the following description or illustrated in the drawings. The invention is capable of other embodiments and of being practiced or of being carried out in various ways. Also, the phraseology and terminology used in this application is for the purpose of description and should not be regarded as limiting. The use of “including,” “comprising,” or “having,” “containing,” “involving,” and variations thereof, is meant to encompass the items listed thereafter and equivalents thereof as well as additional items. The term “a” or “an” refers to one or more of an entity.

### BRIEF DESCRIPTION OF THE DRAWINGS

FIG. 1 is a bar chart showing the aggregation rates in terms of percentage of High Molecular Weight species (% HMW) in the exemplary formulations listed in Table 1, measured by SE-UHPLC over a period of 4 weeks at 5° C. The time points (0, 1, 2, 3, and 4 weeks) on the X axis are shown from left to right for each sample.

FIG. 2 is a graph showing the change in amount of % HMW species in the formulations listed in Table 2 through the different steps of a scale-down manufacturing process, as measured by SE-UHPLC.

FIG. 3 is a graph showing the change in amount of % HMW species in the formulations listed in Table 2 over a period of 12 weeks at 5° C., as measured by SE-UHPLC.

FIG. 4 is a graph that shows the projected end-to-end % HMW projection for the expected shelf-life of the 25 mg/mL formulation and the 50 mg/mL formulation at various times; at post formulation time point, 50 mg/mL sample shown was diluted to 25 mg/mL and monitored through 3 years storage alongside 25 mg/mL sample.

FIG. 5 is a bar chart showing % HMW in the 25 mg/mL formulation and the 50 mg/mL formulations, measured by SE-UHPLC over a period of OM, 3M, and projected 24M at 5° C.

FIG. 6 is a graph showing the amount of aggregation (percent high molecular weight, % HMW) over time at 5° C. as measured by size-exclusion chromatography. Glu/Thr (10 mM glutamic acid, 250 mM threonine) showed up to 0.5% HMW less aggregation compared to RP formulation (RPF): 10 mM histidine, 7% w/v sucrose over 104 weeks of stability at 5° C.

FIG. 7 is a graph showing change in aggregation (percent high molecular weight, % HMW) from 0 weeks to 104 weeks at 5° C. as measured by size-exclusion chromatography. Glu/Thr (10 mM glutamic acid, 250 mM threonine; 0.1% HMW) showed less of a change in aggregation compared to RPF (10 mM histidine, 7% w/v sucrose; 0.2% HMW) after 104 weeks of stability at 5° C.

FIG. 8 is a graph showing the amount of aggregation (percent high molecular weight, % HMW) over time at 25° C. as measured by size-exclusion chromatography. Glu/Thr (10 mM glutamic acid, 250 mM threonine) showed up to 0.4% HMW less aggregation compared to RPF (10 mM histidine, 7% w/v sucrose) over 24 weeks of stability at 25° C.

FIG. 9 is a graph showing change in aggregation (percent high molecular weight, % HMW) from 0 weeks to 24 weeks at 25° C. as measured by size-exclusion chromatography. Glu/Thr (10 mM glutamic acid, 250 mM threonine; 0.1% HMW) showed equivalent or less of a change in aggregation compared to RPF (10 mM histidine, 7% w/v sucrose; 0.2% HMW) over 24 weeks of stability at 25° C.

FIG. 10 is a graph showing the amount of aggregation (percent high molecular weight, % HMW) in various buffer-stabilizer combinations as measured by size-exclusion chromatography. Glu-highThr (10 mM glutamic acid, 271 mM threonine) maintained a similar % HMW level to RPF (10 mM histidine, 7% w/v sucrose) over 4 weeks at 25° C. Glu-highThr maintained a lower % HMW than formulations with lower threonine (Glu-lowThr-Glyc, Glu-medThr-Glyc) or no threonine (GluGlyc, Glyc).

FIG. 11 is a graph showing the amount of aggregation (percent high molecular weight, % HMW) in various buffer-stabilizer combinations as measured by size-exclusion chromatography. Glu-highThr (10 mM glutamic acid, 271 mM threonine) maintained a similar % HMW level to RP (10 mM histidine, 7% w/v sucrose) over 8 weeks at 5° C. Glu-highThr maintained a lower % HMW than formulations with lower threonine (Glu-lowThr-Glyc, Glu-medThr-Glyc) or no threonine (GluGlyc, Glyc).

FIG. 12 is a graph showing the amount of aggregation (percent high molecular weight, % HMW) over 4 weeks at 25° C. as measured by size-exclusion chromatography.

FIG. 13 is a graph showing the amount of aggregation (percent high molecular weight, % HMW) in various buffer-stabilizer combinations over 4 weeks at 25° C. as measured by size-exclusion chromatography.

FIG. 14 is a graph showing the amount of aggregation (percent high molecular weight, % HMW) in various buffer-stabilizer combinations over 4 weeks at 25° C. as measured by size-exclusion chromatography.

### DETAILED DESCRIPTION

The anti-PD-1 antibody pharmaceutical formulation for the marketed pembrolizumab product contains a buffer, a stabilizer, and a surfactant for drug stability. However, some buffers are prone to self-degradation under stress conditions, and some surfactants are known to be degraded through oxidation and hydrolysis, which in turn can generate reactive oxygen species (ROS), which can cause oxidative damage to antibody proteins. Therefore, there is a need to develop formulations capable of stabilizing a protein without the use of excipients that may affect the stability of the anti-PD-1 antibody pharmaceutical formulation and do not require the presence of a buffer.

The present disclosure provides stable pharmaceutical formulations comprising an anti-PD-1 antibody (e.g., pembrolizumab) or antigen-binding fragment thereof, and methods of making stable pharmaceutical formulations comprising an anti-PD-1 antibody (e.g., pembrolizumab). Also provided are lyophilized forms of the stable pharmaceutical formulations disclosed herein. In other aspects, the disclosure also provides uses of the formulations disclosed herein and methods of administering these formulations to treat diseases such as cancers and infectious diseases.

“Pembrolizumab” is a humanized antibody used in cancer immunotherapy, marketed under the name KEYTRUDA®. Pembrolizumab targets the PD-1 receptor on lymphocytes. Pembrolizumab is an IgG 4 isotype antibody that blocks the protective mechanism of cancer cells, allowing the immune system to destroy cancer cells. Pembrolizumab was first approved for medicinal use in the United States in 2014 for the treatment of patients with unresectable or metastatic melanoma and disease progression filing ipilimumab and, if BRAF V600 positive, a BRAF inhibitor. In addition, pembrolizumab was approved in the United States between 2014 and 2022 for: treatment of advanced non-small cell lung cancer; treatment of recurrent or metastatic head and neck squamous cell carcinoma; treatment of metastatic non-small cell lung cancer; treatment of classical Hodgkin lymphoma; treatment of metastatic non-squamous non-small cell lung Cancer (NSCLC), irrespective of PD-L1 expression; treatment of locally advanced or metastatic urothelial carcinoma; treatment of any solid tumor with a specific genetic feature, such as mismatch repair deficiency or microsatellite instability; treatment of previously treated patients with recurrent locally advanced or metastatic gastric or gastroesophageal junction cancer whose tumors express PD-L1; treatment of previously treated patients with recurrent or metastatic cervical cancer whose tumors express PD-L1; treatment of refractory or relapsed primary mediastinal large B-cell lymphoma (PMBCL); treatment of patients with metastatic nonsquamous NSCLC with no EGFR or ALK Genomic tumor aberrations; treatment in combination with carboplatin and either paclitaxel or nab-paclitaxel for the first-line treatment of patients with metastatic squamous non-small cell lung cancer (NSCLC); treatment of patients with hepatocellular carcinoma (HCC) who have been previously treated with Sorafenib; treatment of patients with recurrent locally advanced or metastatic Merkel cell carcinoma; the adjuvant treatment of patients with melanoma with involvement of lymph node(s) following complete resection; treatment in combination with axitinib as first-line treatment for patients with advanced renal cell carcinoma (RCC); first-line treatment of head and neck squamous cell carcinoma; treatment of metastatic small cell lung cancer (SCLC); treatment of recurrent locally advanced or metastatic squamous cell carcinoma of the esophagus; treatment in combination with lenvatinib for patients with certain types of endometrial carcinoma; treatment of patients with BCG-unresponsive, high-risk, non-muscle invasive bladder cancer; treatment of patients with recurrent or metastatic cutaneous squamous cell carcinoma (cSCC) that is not curable by surgery or radiation; first-line treatment of patients with unresectable or metastatic MSI-H or dMMR colorectal cancer; treatment of adult patients with relapsed or refractory classical Hodgkin lymphoma (cHL); treatment in combination with chemotherapy for patients with locally recurrent unresectable or metastatic triple-negative breast cancer whose tumors express PD-L1 (CPS≥10); treatment with platinum- and fluoro-pyrimidine-based chemotherapy of certain patients with locally advanced or metastatic esophageal or gastroesophageal junction (GEJ) carcinoma; treatment combined with trastuzumab and chemotherapy as first-line treatment in locally advanced unresectable or metastatic HER2-positive gastric or gastroesophageal junction adenocarcinoma; treatment in combination with lenvatinib for patients with certain types of advanced endometrial carcinoma; treatment of patients with high-risk early-stage triple-negative breast cancer in combination with chemotherapy as neoadjuvant treatment, then continued as single agent as adjuvant treatment after surgery; treatment in combination with lenvatinib for first-line treatment of adult patients with advanced renal cell carcinoma (RCC); treatment in combination with chemotherapy, with or without bevacizumab, treatment for patients with persistent, recurrent or metastatic cervical cancer whose tumors express PD-L1 (CPS≥1); treatment as adjuvant therapy for certain patients with renal cell carcinoma (RCC) following surgery; and for treatment of patients with MSI-H/dMMR advanced endometrial carcinoma, who have disease progression following prior systemic therapy in any setting and are not candidates for curative surgery or radiation. Pembrolizumab may be used alone or in combination with chemotherapeutic agents. Pembrolizumab may be administered by intravenous or subcutaneous injection.

Pembrolizumab can be produced by general methods known in the art. For example, U.S. Pat. No. 9,834,605, which is incorporated herein by reference in its entirety, describes methods that one of ordinary skill in the art can use to prepare pembrolizumab antibodies. For example, pembrolizumab can be prepared by recombinant expression of immunoglobulin light and heavy chain genes in a host cell.

Pembrolizumab is indicated for melanoma, lung cancer including non-small cell lung cancer (NSCLC), head and neck cancer including head and neck squamous cell cancer (HNSCC), Hodgkin's lymphoma, including classical Hodgkin Lymphoma (cHL), B-cell lymphoma, including mediastinal large B-cell lymphoma (PMBCL), urothelial carcinoma, renal cell carcinoma, gastric cancer, microsatellite instability-high (MSI-H) cancer, mismatch repair deficient (dMMR) cancer, including MSI-H and dMMR colorectal cancer; and can be used to treat solid cancer, cervical cancer, liver cancer, Merkel cell carcinoma (MCC), esophageal cancer, hepatocellular carcinoma (HCC), endometrial carcinoma, tumor mutational burden-high (TMB-H) cancer, cutaneous squamous cell carcinoma (cSCC), triple-negative breast cancer (TNBC), and the like. The pembrolizumab may also include a biosimilar of the active pembrolizumab present in commercially available Keytruda. Pembrolizumab includes a heavy chain variable region comprising SEQ ID Nos: 1 to 3, and a light chain variable region comprising SEQ ID Nos: 4 to 6. Pembrolizumab has a heavy chain CDR1 having the amino acid sequence of SEQ ID NO: 1 (NYYMY), a heavy chain CDR2 having the amino acid sequence of SEQ ID NO: 2 (GINPSNGGTNFNEKFK), and a heavy chain CDR3 having the amino acid sequence of SEQ ID NO: 3 (RDYRFDMGFDY), and a light chain CDR1 having an amino acid sequence of 4 (RASKGVSTSGYSYLH), a light chain CDR2 having an amino acid sequence of SEQ ID NO: 5 (LASYLES), and a light chain CDR3 having an amino acid sequence of SEQ ID NO: 6 (QHSRDLPLT). Pembrolizumab comprises a heavy chain variable region having the amino acid sequence of SEQ ID NO: 7 (VQLVQSGVEVKKPGASVKVSCKASGYTFTNYYMYWVRQAPGQGLEWMGGINPSN GGTNFNEKFKNRVTLTTDSSTTTAYMELKSLQFDDTAVYYCARRDYRFDMGFDYW GQGTTVTVSSASTKGPSVFPLAPCSRSTSESTAALGCLVKDYFPEPVTVSWNSGALTS GVHTFPAVLQSSGLYSLSSVVTVPSSSLGTKTYTCNVDHKPSNTKVDKRVESKYGPP CPPCPAPEFLGGPSVFLFPPKPKDTLMISRTPEVTCVVVDVSQEDPEVQFNWYVDGV EVHNAKTKPREEQFNSTYRVVSVLTVLHQDWLNGKEYKCKVSNKGLPSSIEKTISKA KGQPREPQVYTLPPSQEEMTKNQVSLTCLVKGFYPSDIAVEWESNGQPENNYKTTPP VLDSDGSFFLYSRLTVDKSRWQEGNVFSCSVMHEALHNHYTQKSLSLSLGK) and light chain variable region having the amino acid sequence of SEQ ID NO: 8 (IVLTQSPATLSLSPGERATLSCRASKGVSTSGYSYLHWYQQKPGQAPRLLIYLASYL ESGVPARFSGSGSGTDFTLTISSLEPEDFAVYYCQHSRDLPLTFGGGTKVEIKRTVAAP SVFIFPPSDEQLKSGTASVVCLLNNFYPREAKVQWKVDNALQSGNSQESVTEQDSKD STYSLSSTLTLSKADYEKHKVYACEVTHQGLSSPVTKSFNRGEC).

As used herein, a “biosimilar,” particularly a pembrolizumab biosimilar, is a biological product that is highly similar to pembrolizumab notwithstanding minor differences in clinically inactive components, with no clinically meaningful differences between the biological product and pembrolizumab in terms of safety, purity, and potency of the product. As used herein, the term “pembrolizumab” includes a pembrolizumab biosimilar.

As used herein, an “antibody” is a heterotetrameric glycoprotein of about 150,000 daltons, composed of two identical and substantially full-length light (L) chains and two identical and substantially full-length heavy (H) chains. Each light chain is linked to a heavy chain by one covalent disulfide bond, while the number of disulfide linkages varies between the heavy chains of different immunoglobulin isotypes. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Chothia et al. J. Mol. Biol. 186:651 (1985); Novotny and Haber, Proc. Natl. Acad. Sci. U.S.A. 82:4592 (1985); Chothia et al., Nature 342:877-883 (1989)).

As used herein, an “antibody fragment” includes fragments of an antibody that bind the target antigen. Examples of antibody fragments include Fab, Fab′, F(ab′)₂, and Fv fragments.

As used herein, the term “pharmaceutically effective amount” or “effective amount” means an amount whereby sufficient therapeutic composition or formulation is introduced to a patient to treat a disease or condition.

As used herein, the term “about” when modifying the quantity (e.g., mM or M) of a substance or composition, the percentage (v/v or w/v) of a formulation component, the pH of a solution/formulation or the value of a parameter characterizing a step in a method, or the like refers to variation in the numerical quantity that can occur, for example, through typical measuring, handling and sampling procedures involved in the preparation, characterization and/or use of the substance or composition through instructional error in these procedures, through differences in the manufacture, source or purity of ingredients employed to make or use the compositions or carry about the procedures and the like. In certain embodiments, “about” can mean a variation of ±0.1%, 0.5%, 1%, 2%, 3%, 4%, 5% or 10%.

As used herein, “x % (w/v)” is equivalent to x g/100 ml (for example, 5% w/v equals 50 mg/ml).

As used herein, the term “pharmaceutical formulation” refers to preparations which are in such form to permit the active ingredients to be effective. The term “formulation” and “pharmaceutical formulation” are used interchangeably throughout.

As used herein, “stable formulation” is one in which the active ingredient, for example the protein, therein essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Stability can be measured at a selected temperature for a selected time period. For example, in one embodiment, a stable formulation is a formulation with no significant changes observed at a refrigerated temperature (2-8° C.) for at least 12 months, such as for at least 18 months or at least 24 months. In another embodiment, a stable formulation is a formulation with no significant changes observed at a refrigerated temperature (2-8° C.) for at least 18 months. In another embodiment, a stable formulation is a formulation with no significant changes observed at refrigerated temperature (2-8° C.) for at least 60 months. A stable formulation with no significant changes is one in which no more than 10%, preferably 5%, of antibody monomer is degraded as measured by SEC-HPLC or SEC-UHPLC. A stable formulation is colorless, or clear to slightly opalescent by visual analysis. The concentration, pH and osmolality of the stable formulation have no more than +/−10% change. Potency is typically within 60-140%, preferably 80-120% of the control or reference. A stable formulation has no more than 10%, such as 5% or less of clipping of the antibody is observed, e.g., % low molecular weight species as determined, for example, by HP-SEC. A stable formulation has no more than 5% of aggregation of the antibody observed, e.g., % of high molecular weight species as determined, for example, by HP-SEC.

In embodiments, stable pharmaceutical formulations described herein include an anti-PD-1 antibody (e.g., pembrolizumab) and one or more excipients selected such that the formulation demonstrates characteristics suitable for use as a pharmaceutical composition. A formulation suitable for use as a pharmaceutical composition generally exhibits a low amount of high molecular weight species (referred to herein as “HMWS” or “HMW”), such as aggregates and dimers, and/or also exhibits a low degree of oxidation over time. A suitable formulation may also exhibit a minimal amount of sub-visible particles (e.g., <6000 per container for particles having a diameter of ≥10 μm and <600 per container for particles having a diameter ≥25 μm) and/or non-spherical particles (e.g., particles having an aspect ratio of ≥5 μm). High amounts of HMWS, oxidation, and/or particles may impact the shelf-life, safety and/or potency of a formulation. In some embodiments, a stable formulation has less than 5%, such as less than 1%, less than 0.1%, less than 0.09%, less than 0.08%, less than 0.07%, less than 0.06%, less than 0.05%, less than about 0.04%, less than about 0.03%, less than 0.02%, such as between 0.01% and 0.08%, between 0.03% and 0.07%, about between 0.04% and 0.06% change in HMW species, such as 0.01%, 0.02%, 0.03%, 0.04%, 0.05%, 0.06%, 0.07%, 0.08%, 0.09% change in HMW species for at least 1 month, at least 2 months, at least 3 months, such as 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 36 months, 48 months or 60 months at 2-8° C., pH 5.2-5.8, such as pH 5.5. Stable pharmaceutical formulations comprising an anti-PD-1 antibody (e.g., pembrolizumab) are described in the embodiments set forth below.

In some embodiments, the stable pharmaceutical formulations described herein are aqueous formulations. As used herein, an “aqueous” formulation contains water. Aqueous formulations can be in a liquid state or a frozen state, and preferably are liquid formulations.

As used herein, a “stable” formulation refers to a formulation that demonstrates stability sufficient to permit administration to a patient. For example, a stable formulation may demonstrate long-term stability, such as stability upon storage for 3 years after manufacturing. Stability of a formulation may, for example, be assessed by growth of high molecular weight species over time. When stability is assessed by increase of HMWS over time, a stable formulation may demonstrate less than about 5% increase in HMWS species aggregation as measured by Size Exclusion-High Performance Liquid Chromatography (SE-HPLC) and/or Size Exclusion-UltraHigh Performance Liquid Chromatography (SE-UHPLC) after storage at 2-8° C., such as 5° C., pH 5.2-5.8, pH 5.3-5.8, including pH 5.5, for at least 4 weeks, 8 weeks, 12 weeks, 24 weeks, 36 weeks, 72 weeks, 104 weeks. When stability is assessed by increase of HMWS over time, a stable formulation may demonstrate less than about 5% increase in HMWS species as measured by SE-HPLC after storage at 2-8° C., pH 5.5, for at least 1 month, at least 2 months, at least 3 months, at least 6 months, at least 9 months, at least 12 months, at least 18 months, at least 24 months, at least 36 months, such as between 3 and 36 months, including 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 36 months, 48 months, and 60 months.

The level of aggregation can be evaluated using methods known to one of ordinary skill in the art including SE-HPLC, Size E (SE-HPLC), SE-UHPLC, Cation Exchange chromatography (CEX), and/or reduced Capillary Electrophoresis (rCE). In some embodiments, SE-HPLC is used to monitor monoclonal antibody aggregation. SE-HPLC is used to determine the HMW species amount and monitor the change in % HMW species over a selected period of time in an anti-PD-1 formulation, such as one or more disclosed pembrolizumab formulations. SE-HPLC is performed by the techniques known to one of skill in the art including those disclosed herein as well as in A. E. Hamielec, S. T, Balke, B. P. Leclair & S. L. Pearce; Ind. Eng. Chem., Prod. Res. Dev., 8,54 (1969), Z. Grubistic, R. Rempp & H. Benoit; J. Polymer Sci., Part B, 5, 753 (1967) “Polymer Handbook”, J. Brandrup, E. H. Immersut: John Wiley & Sons Publisher. Data Reduction in Multidetector Size Exclusion Chromatography, Y Brun J. Liq. Chrom & Rel. Technology, 21 (13), 1979-2015 (1998); Modern Size-Exclusion Liquid Chromatography, W. W. Yau, J. J. Kirkland, D. D. Bly John Wiley & Sons, Inc., New York, 1979, each of which is hereby incorporated by reference in its entirety.

In some embodiments, the pharmaceutical formulations described herein comprise pembrolizumab and one or more (typically, one, two, three, four or five) excipients as described herein and are self-buffering. In some embodiments, the pharmaceutical formulations described herein comprise an anti-PD-1 antibody (e.g., pembrolizumab), one or more stabilizing reagents, and one or more additional excipients as described herein.

As used herein, an “excipient” is a component of a formulation other than water and other than the active agent (e.g., an anti-PD-1 antibody) added to the formulation. Examples of excipients include buffers, stabilizers such as amino acids and amino acid derivatives, polyethylene glycols and polyethylene glycol derivatives, polyols, acids, amines, disaccharides or disaccharide derivatives, polysaccharides or polysaccharide derivatives, salts, and surfactants; and pH-adjusting agents.

In some embodiments, disclosed herein are pharmaceutical formulations including an anti-PD-1 antibody (e.g., pembrolizumab) which are self-buffering and do not include any buffering agents or are in an environment that removes strong buffering capacity, e.g., pH 5.5 for L-glutamic acid. These formulations additionally include one or more (such as one, two, three, four, or five) excipients as described herein.

Examples of amino acids and amino acid derivatives that can be used as excipients include L-glutamic acid (e.g., at a concentration of about 5 mM to about 50 mM, about 5 mM to about 25 mM, about 10 mM to about 20 mM, about 5 mM to about 15 mM, about 7 mM to about 12 mM, about 9 mM to about 11 mM, about 5 mM, about 10 mM, about 15 mM, about 20 mM, and/or about 25 mM, including 5 mM to 50 mM, 5 mM to 25 mM, 10 mM to 20 mM, 5 mM to about 15 mM, 7 mM to 12 mM, 9 mM to 11 mM, such as 5 mM, 10 mM, 15 mM, 20 mM, and/or 25 mM), L-threonine (e.g., at a concentration of about 0.1 to about 450 mM, about 100 mM to about 300 mM, about 200 mM to about 280 mM, about 250 mM to about 275 mM, about 100 mM, about 200 mM, about 225 mM, about 250 mM, about 271 mM, about 300 mM, about 350 mM, about 400 mM, about 450 mM, including 0.1 to 450 mM, 100 mM to 300 mM, 200 mM to 280 mM, 250 mM to 275 mM, such as 100 mM, 200 mM, 225 mM, 250 mM, 271 mM, 300 mM, 350 mM, 400 mM, and/or 450 mM), N-acetyl arginine (e.g., at a concentration of about 0.1 mM to about 450 mM and/or about 90 mM to about 150 mM), citrulline (e.g., at a concentration of about 0.1 mM to about 450 mM and/or about 100 mM to about 150 mM), sarcosine (e.g., at a concentration of about 0.1 mM to about 450 mM and/or about 100 mM to about 150 mM), N-acetyl proline (e.g., at a concentration of about 0.1 mM to about 450 mM, about 100 mM to about 150 mM, about 100 mM to about 300 mM, about 200 mM to about 280 mM, about 250 mM to about 275 mM, about 100 mM, about 200 mM, about 225 mM, about 250 mM, about 271 mM, about 300 mM, about 350 mM, about 400 mM, and/or about 450 mM, including 0.1 mM to 450 mM, 100 mM to 300 mM, 200 mM to 280 mM, and/or 250 mM to 275 mM, such as 100 mM, 200 mM, 225 mM, 250 mM, 271 mM, 300 mM, 350 mM, 400 mM, and/or 450 mM), N-acetyl ornithine (e.g., at a concentration of about 0.1 mM to about 450 mM and/or about 100 mM to about 150 mM), ornithine (e.g., at a concentration of about 0.1 mM to about 450 mM and/or about 100 mM to about 150 mM), beta-alanine (e.g., at a concentration of about 0.1 mM to about 450 mM and/or about 100 mM to about 150 mM), alanine (e.g., at a concentration of about 0.1 mM to about 450 mM and/or about 80 mM to about 120 mM, about 100 mM to about 300 mM, about 200 mM to about 280 mM, about 250 mM to about 275 mM, about 100 mM, about 200 mM, about 225 mM, about 250 mM, about 271 mM, about 300 mM, about 350 mM, about 400 mM, and/or about 450 mM, including 0.1 mM to 450 mM, 100 mM to 300 mM, 200 mM to 280 mM, 250 mM to 275 mM, such as 100 mM, 200 mM, 225 mM, 250 mM, 271 mM, 300 mM, 350 mM, 400 mM, and/or 450 mM), asparagine (e.g., at a concentration of about 0.1 mM to about 450 mM, about 80 mM to about 120 mM, about 100 mM to about 300 mM, about 200 mM to about 280 mM, about 250 mM to about 275 mM, about 100 mM, about 200 mM, about 225 mM, about 250 mM, about 271 mM, about 300 mM, about 350 mM, about 400 mM, and/or about 450 mM, including 0.1 mM to 450 mM, 100 mM to 300 mM, 80 mM to 120 mM, 200 mM to 280 mM, 250 mM to 275 mM, 80 mM, 90 mM, 100 mM, 110 mM, 120 mM, 130 mM, 140 mM, 150 mM, 200 mM, 225 mM, 250 mM, 271 mM, 300 mM, 350 mM, 400 mM, and/or 450 mM), isoleucine (e.g., at a concentration of about 0.1 mM to about 450 mM, about 80 mM to about 120 mM, about 100 mM to about 300 mM, about 200 mM to about 280 mM, about 250 mM to about 275 mM, about 100 mM, about 200 mM, about 225 mM, about 250 mM, about 271 mM, about 300 mM, about 350 mM, about 400 mM, and/or about 450 mM, including 0.1 mM to 450 mM, 100 mM to 300 mM, 80 to 120 mM, 200 mM to 280 mM, 250 mM to 275 mM, 80 mM, 90 mM, 100 mM, 110 mM, 120 mM, 130 mM, 140 mM, 150 mM, 200 mM, 225 mM, 250 mM, 271 mM, 300 mM, 350 mM, 400 mM, or 450 mM), serine (e.g., at a concentration of about 0.1 mM to about 450 mM, about 80 mM to about 120 mM, about 100 mM to about 300 mM, about 200 mM to about 280 mM, about 250 mM to about 275 mM, about 100 mM, about 200 mM, about 225 mM, about 250 mM, about 271 mM, about 300 mM, about 350 mM, about 400 mM, about 450 mM, including 0.1 to 450 mM, 100 mM to 300 mM, 80 to 120 mM, 200 mM to 280 mM, 250 mM to 275 mM, including 80 mM, 90 mM, 100 mM, 110 mM, 120 mM, 130 mM, 140 mM, 150 mM, 200 mM, 225 mM, 250 mM, 271 mM, 300 mM, 350 mM, 400 mM, or 450 mM), aspartic acid (e.g., at a concentration of about 0.1 mM to about 450 mM, about 10 mM to about 30 mM, about 100 mM to about 300 mM, about 200 mM to about 280 mM, about 250 mM to about 275 mM, about 100 mM, about 200 mM, about 225 mM, about 250 mM, about 271 mM, about 300 mM, about 350 mM, about 400 mM, about 450 mM, including 0.1 mM to 450 mM, 10 mM to 30 mM, 100 mM to 300 mM, 200 mM to 280 mM, 250 mM to 275 mM, such as 10 mM, 20 mM, 30 mM, 40 mM, 50 mM, 60 mM, 70 mM, 80 mM, 90 mM, 100 mM, 200 mM, 225 mM, 250 mM, 271 mM, 300 mM, 350 mM, 400 mM, 450 mM), creatine (e.g., at a concentration of about 0.1 mM to about 450 mM, about 15 mM to about 35 mM, about 100 mM to about 300 mM, about 200 mM to about 280 mM, about 250 mM to about 275 mM, about 100 mM, about 200 mM, about 225 mM, about 250 mM, about 271 mM, about 300 mM, about 350 mM, about 400 mM, about 450 mM, including 0.1 to 450 mM, 10 mM to 30 mM, 100 mM to 300 mM, 200 mM to 280 mM, 250 mM to 275 mM, such as 10 mM, 20 mM, 30 mM, 40 mM, 50 mM, 60 mM, 70 mM, 80 mM, 90 mM, 100 mM, 150 mM, 200 mM, 225 mM, 250 mM, 271 mM, 300 mM, 350 mM, 400 mM, 450 mM), glutamine (e.g., at a concentration of about 0.1 mM to about 450 mM, about 40 to about 60 mM, about 40 mM, about 50 mM, about 60 mM, about 70 mM, about 80 mM, about 90 mM, about 100 mM to about 300 mM, about 200 mM to about 280 mM, about 250 mM to about 275 mM, about 100 mM, about 200 mM, about 225 mM, about 250 mM, about 271 mM, about 300 mM, about 350 mM, about 400 mM, about 450 mM, including 0.1 to 450 mM, 40 mM to 60 mM, 100 mM to 300 mM, 200 mM to 280 mM, 250 mM to 275 mM, such as 40 mM, 50 mM, 60 mM, 70 mM, 80 mM, 90 mM, 100 mM, 150 mM, 200 mM, 225 mM, 250 mM, 271 mM, 300 mM, 350 mM, 400 mM, and/or 450 mM), phenylalanine (e.g., at a concentration of about 0.1 mM to about 450 mM, about 40 mM to about 60 mM, about 40 mM, about 50 mM, about 60 mM, about 70 mM, about 80 mM, about 90 mM, about 100 mM to about 300 mM, about 200 mM to about 280 mM, about 250 mM to about 275 mM, about 100 mM, about 200 mM, about 225 mM, about 250 mM, about 271 mM, about 300 mM, about 350 mM, about 400 mM, and/or about 450 mM, including 0.1 to 450 mM, 40 to 60 mM, 100 mM to 300 mM, 200 mM to 280 mM, 250 mM to 275 mM, such as 40 mM, 50 mM, 60 mM, 70 mM, 80 mM, 90 mM, 100 mM, 200 mM, 225 mM, 250 mM, 271 mM, 300 mM, 350 mM, 400 mM, and/or 450 mM), tryptophan (e.g., at a concentration of about 0.1 mM to about 450 mM, about 15 mM to about 35 mM, about 100 mM to about 300 mM, about 200 mM to about 280 mM, about 250 mM to about 275 mM, about 100 mM, about 200 mM, about 225 mM, about 250 mM, about 271 mM, about 300 mM, about 350 mM, about 400 mM, and/or about 450 mM, including 0.1 to 450 mM, 15 mM to 35 mM, 100 mM to 300 mM, 80 to 120 mM, 200 mM to 280 mM, 250 mM to 275 mM, including 15 mM, 20 mM 25 mM, 30 mM, 35 mM, 40 mM, 45 mM, 50 mM, 60 mM, 70 mM, 80 mM, 90 mM, 100 mM, 110 mM, 120 mM, 130 mM, 140 mM, 150 mM, 200 mM, 225 mM, 250 mM, 271 mM, 300 mM, 350 mM, 400 mM, and/or 450 mM), and arginine-HCl (e.g., at a concentration of about 0.1 mM to about 450 mM, about 80 mM to about 120 mM, about 100 mM to about 300 mM, about 200 mM to about 280 mM, about 250 mM to about 275 mM, about 100 mM, about 200 mM, about 225 mM, about 250 mM, about 271 mM, about 300 mM, about 350 mM, about 400 mM, about 450 mM, including 0.1 to 450 mM, 100 mM to 300 mM, 80 to 120 mM, 200 mM to 280 mM, and/or 250 mM to 275 mM, including 80 mM, 90 mM, 100 mM, 110 mM, 120 mM, 130 mM, 140 mM, 150 mM, 200 mM, 225 mM, 250 mM, 271 mM, 300 mM, 350 mM, 400 mM, and/or 450 mM). In some embodiments, a high concentration of L-threonine, such as greater than 100 mM L-threonine, including between 100 mM and 450 mM, 100 mM and 300 mM, 100 mM and 271 mM, 150 mM and 300 mM, 150 mM and 271 mM, 200 mM and 300 mM, 200 mM and 271 mM, 225 mM and 275 mM, 225 mM and 271 mM, 250 mM and 275 mM, 250 mM and 271 mM, including 100 mM, 125 mM, 150 mM, 175 mM, 200 mM, 225 mM, 250 mM, 251 mM, 252 mM, 253 mM, 254 mM, 255 mM, 256 mM, 257 mM, 258 mM, 259 mM, 260 mM, 261 mM, 262 mM, 263 mM, 264 mM, 265 mM, 266 mM, 267 mM, 268 mM, 269 mM, 270 mM, 271 mM, 272 mM, 273 mM, 274 mM, 275 mM, 276 mM, 277 mM, 278 mM, 279 mM, 280 mM, 281 mM, 282 mM, 283 mM, 284 mM, 285 mM, 286 mM, 289 mM, 290 mM, 291 mM, 292 mM, 293 mM, 294 mM, 295 mM, 296 mM, 297 mM, 298 mM, 299 mM, 300 mM, 325 mM, 350 mM, 375 mM, 400 mM, 425 mM or 450 mM is used.

Excipients can also include suitable polyethylene glycols and polyethylene glycol derivatives, including but not necessarily limited to PEG 15 hydroxystearate (e.g., at a concentration of about 0.1% (w/v) to about 20% (w/v) or about 3% (w/v) to about 6% (w/v)), PEG 3350 (e.g., at a concentration of about 0.1% (w/v) to about 30% (w/v) or about 1% (w/v) to about 7% (w/v)), PEG 200 (e.g., at a concentration of about 0.1% (w/v) to about 10% (w/v) or about 0.6% (w/v) to about 4.8% (w/v)), PEG 600 (e.g., at a concentration of about 0.1% (w/v) to about 30% (w/v) or about 1.2% (w/v) to about 14.5% (w/v)), and PEG 400 (e.g., at a concentration of about 0.1% (w/v) to about 20% (w/v) or about 0.3% (w/v) to about 1.5% (w/v)).

Excipients can also include suitable polyols, including but not necessarily limited to inositol (e.g., at a concentration of about 0.1 to about 450 mM or about 150 to about 210 mM), glycerol (also referred to as glycerin) (e.g., at a concentration of about 0.1% (w/v) to about 15% (w/v) or about 0.5% (w/v) to about 1% (w/v)), sucrose (e.g., at a concentration of about 0.10% (w/v) to about 15% (w/v), about 4% (w/v) to about 10% (w/v), about 6% (w/v) to about 8.5% (w/v), about 6.2% (w/v) to about 7.3% (w/v), about 4% (w/v) to about 9% (w/v), about 6.5% (w/v), about 6.8% (w/v), about 6.9% (w/v), about 7.4% (w/v), or about 9% (w/v)), and sorbitol (e.g., at a concentration of about 0.1% (w/v) to about 10% (w/v), about 3% (w/v) to about 5% (w/v), and/or about 4% (w/v)). Examples of suitable acids include glycolic acid (e.g., at a concentration of about 0.1 to about 300 mM or about 50 to about 70 mM), pyrollidone carboxylic acid (PCA) (e.g., at a concentration of about 0.1% (w/v) to about 15% (w/v) or about 0.05% (w/v) to about 2% (w/v)), medronic acid (e.g., at a concentration of about 0.1 to about 450 mM or about 100 to about 150 mM), benzene sulfonic acid (e.g., at a concentration of about 0.1 to about 300 mM or about 60 to about 90 mM), and methane sulfonic acid (MSA) (e.g., at a concentration of about 0.1 to about 150 mM, about 0.1 to about 50 mM, and/or about 10 to about 30 mM). Examples of suitable amines include monoethanolamine hydrochloride (MEA-HCl) (e.g., at a concentration of about 0.1 to about 150 mM or about 0.1 to about 40 mM), monoethanolamine (MEA) (e.g., at a concentration of about 0.1 to about 300 mM, about 0.1 to about 50 mM, and/or about 30 to about 160 mM), and triethanolamine (TEA) (e.g., at a concentration of about 0.1 to about 170 mM or about 30 to about 150 mM).

Excipients can also include suitable polysaccharides or polysaccharide derivatives, including but not necessarily limited to hyaluronic acid (e.g., at a concentration of about 0.05% (w/v) to about 2.5% (w/v) or about 0.1% (w/v) to about 0.05% (w/v)), sodium carboxymethylcellulose (NaCMC) (e.g., at a concentration of about 0.1% (w/v) to about 15% (w/v) or about 0.1% (w/v) to about 2% (w/v)), and dextran (e.g., at a concentration of about 0.1% (w/v) to about 15% (w/v) or about 8% (w/v) to about 12% (w/v)).

Examples of suitable surfactants include Pluronic F68 (e.g., at a concentration of about 0.001% (w/v) to about 10% (w/v), about 0.005% (w/v) to about 1% (w/v), about 0.05% (w/v) to about 0.4% (w/v), about 0.05% (w/v) to about 0.1% (w/v), about 0.01% (w/v) to about 0.2% (w/v), about 0.03% (w/v) to about 0.06% (w/v), about 0.01% (w/v), about 0.05% (w/v), about 0.06% (w/v), and/or about 0.1% (w/v)), Polysorbate 80 (e.g., at a concentration of about 0.001% (w/v) to about 2% (w/v), about 0.005% (w/v) to about 0.1% (w/v), about 0.03% (w/v) to about 0.1% (w/v), about 0.04% (w/v) to about 0.10% (w/v), about 0.02 (w/v), about 0.03% (w/v), about 0.04% (w/v), such as 0.01% (w/v), 0.02% (w/v), 0.03% (w/v), 0.04% (w/v), 0.05% (w/v), 0.06% (w/v), 0.07% (w/v), 0.08% (w/v), 0.09% (w/v), 0.1% (w/v)), Polysorbate 20 (e.g., at a concentration of about 0.001% (w/v) to about 2% (w/v) or about 0.005% (w/v) to about 0.1% (w/v)), Docusate sodium (e.g., at a concentration of about 0.001% (w/v) to about 1% (w/v) or about 0.005% (w/v) to about 0.05% (w/v)), benzalkonium chloride (e.g., at a concentration of about 0.001% (w/v) to about 1% (w/v) or about 0.05% (w/v) to about 0.5% (w/v)), Span 40 (sorbitan monopalmitate) (e.g., at a concentration of about 0.001% (w/v) to about 1% (w/v) or about 0.05% (w/v) to about 0.5% (w/v)), and Triton X-100 (e.g., at a concentration of about 0.001% (w/v) to about 1% (w/v) or about 0.005% (w/v) to about 0.1% (w/v)).

Examples of other suitable excipients include imidazole (e.g., at a concentration of about 0.1% (w/v) to about 15% (w/v) or about 0.5% (w/v) to about 2% (w/v)), taurine (e.g., at a concentration of about 0.1 to about 450 mM or about 100 to about 150 mM), betaine (e.g., at a concentration of about 0.1 to about 450 mM or about 100 to about 150 mM), gelatin (e.g., at a concentration of about 0.1% (w/v) to about 15% (w/v) or about 0.5% (w/v) to about 2% (w/v)), niacinamide (e.g., at a concentration of about 0.1 to about 450 mM or about 100 to about 120 mM), polyvinylpyrrolidone (PVP), for example, 10K PVP, (e.g., at a concentration of about 0.001% (w/v) to about 10% (w/v) or about 0.05% (w/v) to about 2% (w/v)), guanidine hydrochloride (GnHCl) (e.g., at a concentration of about 0.1 to about 150 mM or about 10 to about 30 mM), and ethanol (e.g., at a concentration of about 0.05% (w/v) to about 2.5% (w/v) or about 0.25% (w/v) to about 1% (w/v)).

Optionally, the pharmaceutical formulation has a pH of about 4.8 to about 5.8, such as between pH 5.2 and 5.8, for example, about 4.9 to about 5.6, about 5.0 to about 5.5, about 5.1 to about 5.2, about 5.1 to about 5.4, about 5.1 to about 5.3, about 5.2 to about 5.3, about 5.2 to about 5.8, about 5.3 to about 5.5, about 5.4 to about 5.5, about 5.4 to about 5.6, about 5.5 to about 5.6, about 5.5 to about 5.7, about 5.5 to about 5.8, about 5.6 to about 5.7, about 5.7 to about 5.8, between 4.8 and 5.5, 5.3 and 5.6, 5.4 and 5.6, 5.4 and 5.5, 5.4 and 5.7, 5.5 and 5.6, 5.5 and 5.7, 5.5 and 5.8, such as including, about 5.1, about 5.2 about 5.3, about 5.4, about 5.5, about 5.6, about 5.7 and/or about 5.8, such as 5.1, 5.2, 5.3, 5.4, 5.5, 5.6, 5.7, or 5.8. Optionally, the pH of the stable pharmaceutical formulation is adjusted using a strong acid and/or a strong base including, but not limited to, hydrochloric acid or sodium hydroxide.

In some embodiments, the disclosure provides a stable pharmaceutical formulation comprising: (i) an anti-PD-1 antibody, (ii) a surfactant, and (iii) a stabilizing agent. In some embodiments, the disclosure provides a stable pharmaceutical formulation comprising an anti-PD-1, wherein the formulation does not comprise a buffer or does not comprise an excipient that has strong buffering capacity in the environment, such as at a specific pH. In embodiments, the formulation does not comprise histidine. In embodiments, the formulation does not comprise a disaccharide, such as sucrose or trehalose and/or a buffer, such as histidine, acetic acid or citric acid or any other excipient with strong buffering capacities at pH 5.5 to 5.8, such as pH 5.5. A solution with strong or significant buffering capacity is one in which the pH is ±1 unit from the pKa. In this pH range, appreciable amounts of both conjugate base and conjugate acid species are present in solution. Histidine has 3 pKas—1.5, 6.0, and 9; acetic acid has a pKa of 4.8; and citric acid has 3 pKa 3.1, 4.8 and 6.4. As such, histidine, acetic acid and citric acid have significant buffering capacity at pH 5.5 and are considered to be strong buffering agents at this pH (e.g., pH 5.5 is within ±1 unit from the pKa of each respective molecule).

In some embodiments, formulations described herein comprise an anti-PD-1 antibody thereof at a concentration of about 25 mg/mL, such as 25 mg/mL. In some embodiments, formulations described herein comprise an anti-PD-1 antibody thereof at a concentration of about 50 mg/mL, such as 50 mg/mL. In some embodiments, the concentration of anti-PD-1 antibody in the formulation is about 5 mg/mL to about 200 mg/mL, such as 5 mg/mL to 200 mg/mL. In some embodiments, the concentration of anti-PD-1 antibody in the formulation is about 5 mg/mL to about 500 mg/mL, such as 5 mg/mL to 500 mg/mL. In some embodiments, the concentration of anti-PD-1 antibody in the formulation is about 5 mg/mL, about 10 mg/mL, about 20 mg/mL, about 25 mg/mL, about 30 mg/mL, about 40 mg/mL, about 50 mg/mL, about 60 mg/mL, about 70 mg/mL, about 75 mg/mL, about 80 mg/mL, about 90 mg/mL, about 100 mg/mL, about 110 mg/mL, about 120 mg/mL, about 125 mg/mL, about 130 mg/mL, about 140 mg/mL, about 150 mg/mL, about 160 mg/mL, about 165 mg/mL, about 167 mg/mL, about 170 mg/mL, about 175 mg/mL, about 180 mg/mL, about 190 mg/mL, about 200 mg/mL, about 220 mg/mL, about 240 mg/mL, about 250 mg/mL, about 300 mg/mL, about 350 mg/mL, about 400 mg/mL, about 450 mg/mL or about 500 mg/mL. In some embodiments, the amount of anti-PD-1 antibody comprised in the formulation is between 200 mg/mL and 500 mg/mL, such as about 200 mg/mL, about 250 mg/mL, about 300 mg/mL, about 350 mg/mL, about 400 mg/mL, about 450 mg/mL or about 500 mg/mL. In some embodiments, the amount of anti-PD-1 antibody comprised in the formulation is from about 5 mg/mL to about 10 mg/mL, from about 5 mg/mL to about 20 mg/mL, from about 5 mg/mL to about 30 mg/mL, from about 5 mg/mL to about 40 mg/mL, from about 5 mg/mL to about 50 mg/mL, from about 5 mg/mL to about 60 mg/mL, from about 5 mg/mL to about 75 mg/mL, from about 10 mg/mL to about 30 mg/mL, from about 10 mg/mL to about 40 mg/mL, from about 10 mg/mL to about 50 mg/mL, from about 10 mg/mL to about 75 mg/mL, from about 25 mg/mL to about 50 mg/mL, from about 50 mg/mL to about 200 mg/mL, from about 75 mg/mL to about 200 mg/mL, from about 100 mg/mL to about 200 mg/mL, from about 25 mg/mL to about 175 mg/mL, from about 50 mg/mL to about 175 mg/mL, from about 75 mg/mL to about 175 mg/mL, from about 100 mg/mL to about 175 mg/mL, from about 25 mg/mL to about 150 mg/mL, from about 50 mg/mL to about 150 mg/mL, from about 75 mg/mL to about 150 mg/mL, from about 100 mg/mL to about 150 mg/mL, from about 25 mg/mL to about 125 mg/mL, from about 50 mg/mL to about 125 mg/mL, from about 75 mg/mL to about 125 mg/mL, from about 25 mg/mL to about 100 mg/mL, from about 125 mg/mL to about 175 mg/mL, from about 125 mg/mL to about 200 mg/mL, from about 5 mg/mL to 200 mg/mL, from about 150 mg/mL to about 300 mg/mL, from about 150 mL to about 400 mg/mL, from about 150 mL to about 450 mg/mL, from about 150 mg/mL to about 250 mg/mL, from about 200 mg/mL to about 500 mg/mL, from about 200 mg/mL to about 300 mg/mL, from about 200 mg/mL to 400 mg/mL or from about 200 mg/mL to about 500 mg/mL.

In some embodiments, formulations described herein comprise pembrolizumab at a concentration of 25 mg/mL. In some embodiments, formulations described herein comprise pembrolizumab at a concentration of 50 mg/mL. In some embodiments, the concentration of pembrolizumab in the formulation is about 5 mg/mL to about 200 mg/mL, such as 5 mg/mL to 200 mg/mL. In some embodiments, the concentration of pembrolizumab in the formulation is about 25 mg/mL to about 200 mg/mL. In some embodiments, the concentration of pembrolizumab in the formulation is about 5 mg/mL, about 10 mg/mL, about 20 mg/mL, about 25 mg/mL, about 30 mg/mL, about 40 mg/mL, about 50 mg/mL, about 60 mg/mL, about 70 mg/mL, about 75 mg/mL, about 80 mg/mL, about 90 mg/mL, about 100 mg/mL, about 110 mg/mL, about 120 mg/mL, about 125 mg/mL, about 130 mg/mL, about 140 mg/mL, about 150 mg/mL, about 160 mg/mL, about 165 mg/mL, about 167 mg/mL, about 170 mg/mL, about 175 mg/mL, about 180 mg/mL, about 190 mg/mL, about 200 mg/mL, about 210 mg/mL, about 220 mg/mL, about 230 mg/mL, about 240 mg/mL, about 250 mg/mL, about 300 mg/mL, about 350 mg/mL, about 400 mg/mL, about 450 mg/mL or about 500 mg/mL. In some embodiments, the amount of pembrolizumab comprised in the formulation is from about 5 mg/mL to about 10 mg/mL, from about 5 mg/mL to about 20 mg/mL, from about 5 mg/mL to about 30 mg/mL, from about 5 mg/mL to about 40 mg/mL, from about 5 mg/mL to about 50 mg/mL, from about 5 mg/mL to about 60 mg/mL, from about 5 mg/mL to about 75 mg/mL, from about 10 mg/mL to about 30 mg/mL, from about 10 mg/mL to about 40 mg/mL, from about 10 mg/mL to about 50 mg/mL, from about 10 mg/mL to about 75 mg/mL, from about 50 mg/mL to about 200 mg/mL, from about 75 mg/mL to about 200 mg/mL, from about 100 mg/mL to about 200 mg/mL, from about 25 mg/mL to about 175 mg/mL, from about 50 mg/mL to about 175 mg/mL, from about 75 mg/mL to about 175 mg/mL, from about 100 mg/mL to about 175 mg/mL, from about 25 mg/mL to about 150 mg/mL, from about 50 mg/mL to about 150 mg/mL, from about 75 mg/mL to about 150 mg/mL, from about 100 mg/mL to about 150 mg/mL, from about 25 mg/mL to about 125 mg/mL, from about 50 mg/mL to about 125 mg/mL, from about 75 mg/mL to about 125 mg/mL, from about 25 mg/mL to about 100 mg/mL, from about 125 mg/mL to about 175 mg/mL, from about 125 mg/mL to about 200 mg/mL, from about 5 mg/mL to 200 mg/mL, from about 150 mg/mL to about 300 mg/mL, from about 150 mL to about 400 mg/mL, from about 150 mL to about 450 mg/mL, from about 150 mg/mL to about 250 mg/mL, from about 200 mg/mL to about 500 mg/mL, from about 200 mg/mL to about 300 mg/mL, from about 200 mg/mL to 400 mg/mL or from about 200 mg/mL to about 500 mg/mL.

In some embodiments, the concentration of pembrolizumab in the formulation is 5 mg/mL, 10 mg/mL, 20 mg/mL, 25 mg/mL, 30 mg/mL, 40 mg/mL, 50 mg/mL, 60 mg/mL, 70 mg/mL, 75 mg/mL, 80 mg/mL, 90 mg/mL, 100 mg/mL, 110 mg/mL, 120 mg/mL, 125 mg/mL, 130 mg/mL, 140 mg/mL, 150 mg/mL, 160 mg/mL, 165 mg/mL, 167 mg/mL, 170 mg/mL, 175 mg/mL, 180 mg/mL, 190 mg/mL, 200 mg/mL, 210 mg/mL, 220 mg/mL, 230 mg/mL, 240 mg/mL, 250 mg/mL, 300 mg/mL, 350 mg/mL, 400 mg/mL, 450 mg/mL or 500 mg/mL. In some embodiments, the amount of pembrolizumab comprised in the formulation is from 5 mg/mL to 10 mg/mL, from 5 mg/mL to 20 mg/mL, from 5 mg/mL to 30 mg/mL, from 5 mg/mL to 40 mg/mL, from 5 mg/mL to 50 mg/mL, from 5 mg/mL to 60 mg/mL, from 5 mg/mL to 75 mg/mL, from 10 mg/mL to 30 mg/mL, from 10 mg/mL to 40 mg/mL, from 10 mg/mL to 50 mg/mL, from 10 mg/mL to 75 mg/mL, from 50 mg/mL to 200 mg/mL, from 75 mg/mL to 200 mg/mL, from 100 mg/mL to 200 mg/mL, from 25 mg/mL to 175 mg/mL, from 50 mg/mL to 175 mg/mL, from 75 mg/mL to 175 mg/mL, from 100 mg/mL to 175 mg/mL, from 25 mg/mL to 150 mg/mL, from 50 mg/mL to 150 mg/mL, from 75 mg/mL to 150 mg/mL, from 100 mg/mL to 150 mg/mL, from 25 mg/mL to 125 mg/mL, from 50 mg/mL to 125 mg/mL, from 75 mg/mL to 125 mg/mL, from 25 mg/mL to 100 mg/mL, from 125 mg/mL to 175 mg/mL, from 125 mg/mL to 200 mg/mL, from 5 mg/mL to 200 mg/mL, from 150 mg/mL to 300 mg/mL, from 150 mL to 400 mg/mL, from 150 mL to 450 mg/mL, from 150 mg/mL to 250 mg/mL, from 200 mg/mL to 500 mg/mL, from 200 mg/mL to 300 mg/mL, from 200 mg/mL to 400 mg/mL or from 200 mg/mL to 500 mg/mL

In some embodiments, the stabilizing agent is an amino acid. In some embodiments, the amino acid stabilizing agent is valine. In some embodiments, the amino acid stabilizing agent is L-threonine. In some embodiments, the stabilizing agent is L-glutamic acid. In some embodiments, the amino acid stabilizing agent is proline. In some embodiments, the stabilizing agent is a polyol. In some embodiments, the polyol stabilizing agent is glycerol. In some embodiments, the polyol stabilizing agent is sorbitol. In some embodiments, the polyol stabilizing agent is mannitol. In some embodiments, the stabilizing agent is a disaccharide. In some embodiments, the disaccharide stabilizing agent is trehalose. In some embodiments, the disaccharide stabilizing agent is sucrose. In some embodiments, one or more stabilizing agents, such as two, three, four or more are included in the formulation. In some embodiments, two stabilizing agents are included in the formulation. In some embodiments, L-glutamic acid and L-threonine are included in the formulation as stabilizing agents without trehalose and/or sucrose. In some embodiments, the one or more stabilizing agents is not a disaccharide.

In some embodiments, the formulation comprises glycerol. In some embodiments, the formulation comprises trehalose. In some embodiments, the formulation comprises L-glutamic acid and glycerol. In some embodiments, the L-glutamic acid comprised in the formulation is a stabilizing agent. In some embodiments, the formulation comprises L-glutamic acid and valine. In some embodiments, the formulation comprising L-glutamic acid and valine further comprises glycerol. In some embodiments, the L-glutamic acid or the valine comprised in the formulation, or the combination of both the L-glutamic acid and valine comprised in the formulation, is a buffer. In some embodiments, the formulation comprises L-glutamic acid and L-threonine. In some embodiments, the formulation comprising L-glutamic acid and L-threonine further comprises glycerol. In some embodiments, the formulation comprising L-glutamic acid and L-threonine further comprises polysorbate, such as PS20 or PS80. In some embodiments, the formulation comprising L-glutamic acid and L-threonine further comprises PS80 and not histidine, sucrose and/or trehalose. In some embodiments, the L-glutamic acid and/or the L-threonine comprised in the formulation, or the combination of both the L-glutamic acid and L-threonine comprised in the formulation, and the formulation is at pH 5.2 to 5.8, such as pH 5.5. In some embodiments, the formulation comprises L-glutamic acid and proline. In some embodiments, the formulation comprising L-glutamic acid and proline further comprises glycerol. In some embodiments, the formulation comprises proline and glycerol. In some embodiments, the formulation comprises L-glutamic acid and trehalose. In some embodiments, the formulation comprises L-glutamic acid, trehalose, and glycerol. In some embodiments, the formulation comprises L-glutamic acid, sorbitol, and glycerol. In some embodiments, the formulation comprises L-glutamic acid, mannitol, and glycerol. In some embodiments, the formulation comprises acetic acid, L-glutamic acid, and glycerol.

In some embodiments, the formulation is at pH 5.5 and includes one or more amino acids for stabilizing agents and no amino acids having strong buffering capacity at pH 5.5. For example, the pka of the side chain of L-glutamic acid is about 4.2 and the molecule does not have significant buffering capacity at a pH of about 5.5, including pH 5.5. As such, L-glutamic acid is not a strong buffering agent at this pH and in any of the disclosed formulations with L-glutamic acid, with a pH of 5.5.

In some embodiments, self-buffering formulations are disclosed comprising a protein, particularly pharmaceutically acceptable formulations comprising a pharmaceutical protein, that are buffered by the protein itself, that do not require additional buffering agents to maintain a desired pH, and in which the protein is substantially the only buffering agent (i.e., other ingredients, if any, do not act substantially as buffering agents in the formulation).

In some embodiments, a self-buffering formulation is one in which the protein provides about 70-85% of buffering capacity at pH 5.5 to 6.5, about 40 to 60% of buffering capacity at pH 5.5 to 4.5; the lower protein level assumes 50% solvent activity of His/glutamate side chains. In some embodiments, a pharmaceutical formulation is disclosed with self-buffering capabilities, wherein over a range of plus or minus 1 pH unit from a desired pH, a protein provides at least 55%, 60%, 65%, 70%, 75%, 80%, 85%, 90%, 95%, 97%, 98%, 99%, or 99.5%, including between 70-85% of the buffer capacity. In some embodiments, a pharmaceutical formulation is disclosed in which pembrolizumab provides at least 55%, 60%, 65%, 70%, 75%, 80%, 85%, 90%, 95%, 97%, 98%, 99%, or 99.5% of the buffer capacity at a given pH, such as at pH 5.5 to 6.5, and wherein the remainder of the buffering capacity is provided by one or more additional excipients, such as one or more amino acids, such as glutamic acid, including 5 to 25 mM L-glutamic acid, such as 10 mM glutamic acid. In some embodiments, this formulation also includes L-threonine, such as 100 to 300 mM L-threonine, including 250 mM L-threonine, and polysorbate, such as PS80 (e.g., 0.01% to 0.1% w/v PS80).

In some embodiments, a pharmaceutical formulation with self-buffering capabilities is disclosed wherein over the range of plus or minus 1 pH unit from the pH of the formulation, the buffer capacity of the protein, such as pembrolizumab, is at least 1.00 or 1.50 or 1.63 or 2.00 or 3.00 or 4.00 or 5.00 or 6.50 or 8.00 or 10.0 or 15.0 or 20.0 or 30.0 or 40.0 or 50.0 or 75.0 or 100 or 125 or 150 or 200 or 250 or 300 or 350 or 400 or 500 or 700 or 1,000 mEq per liter per pH unit, such as at 1.00, 1.50, 1.63, 2.00, 3.00, 5.0, 10.0, or 20.0.

It is contemplated that in any of the formulations disclosed herein, the concentration of the protein, such as pembrolizumab, is between about 5 and 500 mg/ml, or 20 and 300, or 20 and 250, or 20 and 200, or 20 and 150 mg/ml, such as between approximately 20 and 400 mg/ml, between approximately 20 and 250, between approximately 20 and 150 mg/ml, including 5 mg/mL, 10 mg/mL, 20 mg/mL, 25 mg/mL, 30 mg/mL, 40 mg/mL, 50 mg/mL, 60 mg/mL, 70 mg/mL, 75 mg/mL, 80 mg/mL, 90 mg/mL, 100 mg/mL, 110 mg/mL, 120 mg/mL, 125 mg/mL, 130 mg/mL, 140 mg/mL, 150 mg/mL, 160 mg/mL, 165 mg/mL, 167 mg/mL, 170 mg/mL, 175 mg/mL, 180 mg/mL, 190 mg/mL, 200 mg/mL, 210 mg/mL, 220 mg/mL, 230 mg/mL, 240 mg/mL, 250 mg/mL, 300 mg/mL, 350 mg/mL, 400 mg/mL, 450 mg/mL or 500 mg/mL. In some embodiments, the amount of pembrolizumab comprised in the formulation is from 5 mg/mL to 10 mg/mL, from 5 mg/mL to 20 mg/mL, from 5 mg/mL to 30 mg/mL, from 5 mg/mL to 40 mg/mL, from 5 mg/mL to 50 mg/mL, from 5 mg/mL to 60 mg/mL, from 5 mg/mL to 75 mg/mL, from 10 mg/mL to 30 mg/mL, from 10 mg/mL to 40 mg/mL, from 10 mg/mL to 50 mg/mL, from 10 mg/mL to 75 mg/mL, from 50 mg/mL to 200 mg/mL, from 75 mg/mL to 200 mg/mL, from 100 mg/mL to 200 mg/mL, from 25 mg/mL to 175 mg/mL, from 50 mg/mL to 175 mg/mL, from 75 mg/mL to 175 mg/mL, from 100 mg/mL to 175 mg/mL, from 25 mg/mL to 150 mg/mL, from 50 mg/mL to 150 mg/mL, from 75 mg/mL to 150 mg/mL, from 100 mg/mL to 150 mg/mL, from 25 mg/mL to 125 mg/mL, from 50 mg/mL to 125 mg/mL, from 75 mg/mL to 125 mg/mL, from 25 mg/mL to 100 mg/mL, from 125 mg/mL to 175 mg/mL, from 125 mg/mL to 200 mg/mL, from 5 mg/mL to 200 mg/mL, from 150 mg/mL to 300 mg/mL, from 150 mL to 400 mg/mL, from 150 mL to 450 mg/mL, from 150 mg/mL to 250 mg/mL, from 200 mg/mL to 500 mg/mL, from 200 mg/mL to 300 mg/mL, from 200 mg/mL to 400 mg/mL or from 200 mg/mL to 500 mg/mL.

Exemplary methods for determining the self-buffering capacity of a protein are disclosed in US20130273067A1 which is hereby incorporated by reference in its entirety (see for example, paragraphs [0159]-[0208], Example Section provided in US20130273067A1).

In some embodiments, the formulation includes a buffer. As used herein, a “buffer” is a component of a formulation to which a pharmaceutical agent is added to resist pH change. The buffer is capable of maintaining the pH of the formulation within an acceptable range. In this specification, when a concentration of a buffer is referred to, the stated concentration refers to the molar concentration of the free acid form of the buffer. As stated above, a solution with strong buffering capacity is one in which the pH is ±1 unit from the pKa. In this pH range, appreciable amounts of both conjugate base and conjugate acid species are present in solution. Histidine has 3 pKas—1.5, 6.0, and 9; acetic acid has a pKa of 4.8; and citric acid has 3 pKa 3.1, 4.8 and 6.4. As such, histidine, acetic acid and citric acid have a strong buffering capacity at pH 5.5 and are considered to be strong buffering agents at this pH (e.g., pH 5.5 is within ±1 unit from the pka of each respective molecule). Glutamic acid has three pKa's 2.2, 4.2 and 9.7 and does not have strong buffering capacity at pH 5.5.

Examples of suitable buffers include acetic acid and/or acetate (e.g., at a concentration of about 0.1 mM to about 300 mM, about 2 mM to about 30 mM, about 5 mM to about 50 mM, about 5 mM to about 15 mM, about 10 mM to about 20 mM, about 10 mM to about 30 mM, about 15 mM to about 25 mM, about 30 mM to about 40 mM, about 35 mM to about 45 mM, about 40 mM to about 50 mM, about 10 mM, about 15 mM, about 20 mM, and/or about 25 mM), lactic acid and/or lactate (e.g., at a concentration of about 0.1 mM to about 300 mM, about 2 mM to about 30 mM, about 10 mM to about 30 mM, about 5 mM to about 15 mM, about 7 mM to about 12 mM, about 9 mM to about 11 mM, about 10 mM, about 15 mM, about 20 mM, and/or about 25 mM), and proline (e.g., at a concentration of about 0.1 to about 450 mM, about 50 to about 320 mM, and/or about 50 to about 300 mM). Exemplary buffers for the disclosed formulations do not include histidine.

Examples of other potential buffers include, but are not necessarily limited to, adipate (e.g., at a concentration of about 5 mM to about 50 mM, about 10 mM to about 25 mM, about 15 mM to about 20 mM, about 10 mM, about 15 mM, about 20 mM, and/or about 25 mM), glucuronate (e.g., at a concentration of about 5 mM to about 50 mM, about 10 mM to about 30 mM, about 10 mM, about 15 mM, about 20 mM, and/or about 25 mM), benzoate (e.g., at a concentration of about 5 mM to about 50 mM, about 10 mM to about 30 mM, about 10 mM, about 15 mM, about 20 mM, and/or about 25 mM), and glycolate (e.g., at a concentration of about 5 mM to about 50 mM, about 10 mM to about 30 mM, about 10 mM, about 15 mM, about 20 mM, and/or about 25 mM).

Accordingly, suitable buffers include, but are not necessarily limited to acetate/acetic acid buffers (acetate buffer), lactate/lactic acid buffers (lactated buffer), and proline buffers. Other potential buffers include adipate/adipic acid buffers (adipate buffer), glucuronate/glucuronic acid buffers (glucuronate buffer), benzoate/benzoic acid buffers (benzoate buffer), and glycolate/glycolic acid buffers (glycolate buffer).

In some embodiments, the buffer is a lactic acid buffer. In some embodiments, the buffer is an acetic acid buffer. In some embodiments, the buffer is not a histidine buffer.

In some embodiments, the formulation comprises lactic acid and glycerol. In some embodiments, the lactic acid comprised in the formulation is a buffer. In some embodiments, the formulation comprises lactic acid and L-threonine.

In some embodiments, formulations described herein comprise an amino acid at a concentration from about 2 mM to about 300 mM. In some embodiments, the concentration of an amino acid in a formulation described herein is about 2 mM, about 4 mM, about 5 mM, about 10 mM, about 15 mM, about 20 mM, about 25 mM, about 30 mM, about 40 mM, about 50 mM, about 60 mM, about 70 mM, about 75 mM, about 80 mM, about 90 mM, about 100 mM, about 110 mM, about 115 mM, about 120 mM, about 125 mM, about 130 mM, about 140 mM, about 150 mM, about 160 mM, about 170 mM, about 175 mM, about 180 mM, about 190 mM, about 200 mM, about 210 mM, about 215 mM, about 220 mM, about 225 mM, about 230 mM, about 240 mM, about 250 mM, about 260 mM, about 270 mM, about 271 mM, about 272 mM, about 273 mM, about 274 mM, about 275 mM, about 280 mM, about 290 mM, or about 300 mM. In some embodiments, the concentration of an amino acid in a formulation described herein is from about 2 to about 10 mM, from about 5 to about 10 mM, from about 8 to about 12 mM, from about 8 to about 15 mM, from about 10 to about 20 mM, from about 15 to about 30 mM, from about 20 to about 30 mM, from about 25 to about 40 mM, from about 40 to about 50 mM, from about 40 to about 75 mM, from about 50 to about 100 mM, from about 75 to about 100 mM, from about 100 to about 110 mM, from about 100 to about 115 mM, from about 110 to about 120 mM, from about 115 to about 130 mM, from about 120 to about 130 mM, from about 125 to about 140 mM, from about 140 to about 150 mM, from about 140 to about 175 mM, from about 150 to about 200 mM, from about 175 to about 200 mM, from about 200 to about 215 mM, from about 210 to about 220 mM, from about 215 to about 230 mM, from about 220 to about 230 mM, from about 225 to about 240 mM, from about 240 to about 250 mM, from about 240 to about 275 mM, from about 250 to about 300 mM, or from about 270 to about 300 mM. In some embodiments, the amino acid is valine. In some embodiments, the amino acid is L-glutamic acid. In some embodiments, the amino acid is L-threonine.

In some embodiments, formulations described herein comprise an amino acid at a concentration from 2 mM to 450 mM, such as 2 mM to 300 mM. In some embodiments, the concentration of an amino acid in a formulation described herein is 2 mM, 4 mM, 5 mM, 10 mM, 15 mM, 20 mM, 25 mM, 30 mM, 40 mM, 50 mM, 60 mM, 70 mM, 75 mM, 80 mM, 90 mM, 100 mM, 110 mM, 115 mM, 120 mM, 125 mM, 130 mM, 140 mM, 150 mM, 160 mM, 170 mM, 175 mM, 180 mM, 190 mM, 200 mM, 210 mM, 215 mM, 220 mM, 225 mM, 230 mM, 240 mM, 250 mM, 255 mM, 260 mM, 270 mM, 271 mM, 272 mM, 273 mM, 274 mM, 275 mM, 280 mM, 290 mM, 300 mM, 350 mM, 400 mM, or 450 mM. In some embodiments, the concentration of an amino acid in a formulation described herein is from about 2 to about 10 mM, from about 5 to about 10 mM, from about 8 to about 12 mM, from about 8 to about 15 mM, from about 10 to about 20 mM, from about 15 to about 30 mM, from about 20 to about 30 mM, from about 25 to about 40 mM, from about 40 to about 50 mM, from about 40 to about 75 mM, from about 50 to about 100 mM, from about 75 to about 100 mM, from about 100 to about 110 mM, from 100 to 115 mM, from 110 to 120 mM, from 115 to 130 mM, from 120 to 130 mM, from 125 to 140 mM, from 140 to 150 mM, from 140 to 175 mM, from 150 to 200 mM, from 175 to 200 mM, from 200 to 215 mM, from 210 to 220 mM, from 215 to 230 mM, from 220 to 230 mM, from 225 to 240 mM, from 240 to 250 mM, from 240 to 275 mM, from 250 to 300 mM, from 250 mM to 280 mM, from 250 mM to 271 mM, from 270 to 300 mM, from 250 mM to 400 mM.

In some embodiments, the amino acid is valine. In some embodiments, formulations described herein comprise valine at a concentration from 2 mM to 450 mM, such as 2 mM to 300 mM. In some embodiments, the concentration of valine in a formulation described herein is 2 mM, 4 mM, 5 mM, 10 mM, 15 mM, 20 mM, 25 mM, 30 mM, 40 mM, 50 mM, 60 mM, 70 mM, 75 mM, 80 mM, 90 mM, 100 mM, 110 mM, 115 mM, 120 mM, 125 mM, 130 mM, 140 mM, 150 mM, 160 mM, 170 mM, 175 mM, 180 mM, 190 mM, 200 mM, 210 mM, 215 mM, 220 mM, 225 mM, 230 mM, 240 mM, 250 mM, 255 mM, 260 mM, 270 mM, 271 mM, 272 mM, 273 mM, 274 mM, 275 mM, 280 mM, 290 mM, 300 mM, 350 mM, 400 mM, or 450 mM. In some embodiments, the concentration of valine in a formulation described herein is from about 2 to about 10 mM, from about 5 to about 10 mM, from about 8 to about 12 mM, from about 8 to about 15 mM, from about 10 to about 20 mM, from about 15 to about 30 mM, from about 20 to about 30 mM, from about 25 to about 40 mM, from about 40 to about 50 mM, from about 40 to about 75 mM, from about 50 to about 100 mM, from about 75 to about 100 mM, from about 100 to about 110 mM, from 100 to 115 mM, from 110 to 120 mM, from 115 to 130 mM, from 120 to 130 mM, from 125 to 140 mM, from 140 to 150 mM, from 140 to 175 mM, from 150 to 200 mM, from 175 to 200 mM, from 200 to 215 mM, from 210 to 220 mM, from 215 to 230 mM, from 220 to 230 mM, from 225 to 240 mM, from 240 to 250 mM, from 240 to 275 mM, from 250 to 300 mM, from 250 mM to 280 mM, from 250 mM to 271 mM, from 270 to 300 mM, from 250 mM to 400 mM.

In some embodiments, the amino acid is L-glutamic acid. In some embodiments, formulations described herein comprise L-glutamic acid at a concentration from 2 mM to 450 mM, such as 2 mM to 300 mM. In some embodiments, the concentration of L-glutamic acid in a formulation described herein is 2 mM, 4 mM, 5 mM, 10 mM, 15 mM, 20 mM, 25 mM, 30 mM, 40 mM, 50 mM, 60 mM, 70 mM, 75 mM, 80 mM, 90 mM, 100 mM, 110 mM, 115 mM, 120 mM, 125 mM, 130 mM, 140 mM, 150 mM, 160 mM, 170 mM, 175 mM, 180 mM, 190 mM, 200 mM, 210 mM, 215 mM, 220 mM, 225 mM, 230 mM, 240 mM, 250 mM, 255 mM, 260 mM, 270 mM, 271 mM, 272 mM, 273 mM, 274 mM, 275 mM, 280 mM, 290 mM, 300 mM, 350 mM, 400 mM, or 450 mM. In some embodiments, the concentration of L-glutamic acid in a formulation described herein is from about 2 to about 10 mM, from about 5 to about 10 mM, from about 8 to about 12 mM, from about 8 to about 15 mM, from about 10 to about 20 mM, from about 15 to about 30 mM, from about 20 to about 30 mM, from about 25 to about 40 mM, from about 40 to about 50 mM, from about 40 to about 75 mM, from about 50 to about 100 mM, from about 75 to about 100 mM, from about 100 to about 110 mM, from 100 to 115 mM, from 110 to 120 mM, from 115 to 130 mM, from 120 to 130 mM, from 125 to 140 mM, from 140 to 150 mM, from 140 to 175 mM, from 150 to 200 mM, from 175 to 200 mM, from 200 to 215 mM, from 210 to 220 mM, from 215 to 230 mM, from 220 to 230 mM, from 225 to 240 mM, from 240 to 250 mM, from 240 to 275 mM, from 250 to 300 mM, from 250 mM to 280 mM, from 250 mM to 271 mM, from 270 to 300 mM, from 250 mM to 400 mM.

In some embodiments, the amino acid is L-threonine. In some embodiments, formulations described herein comprise L-threonine at a concentration from 2 mM to 450 mM, such as 2 mM to 300 mM. In some embodiments, the concentration of L-threonine in a formulation described herein is 2 mM, 4 mM, 5 mM, 10 mM, 15 mM, 20 mM, 25 mM, 30 mM, 40 mM, 50 mM, 60 mM, 70 mM, 75 mM, 80 mM, 90 mM, 100 mM, 110 mM, 115 mM, 120 mM, 125 mM, 130 mM, 140 mM, 150 mM, 160 mM, 170 mM, 175 mM, 180 mM, 190 mM, 200 mM, 210 mM, 215 mM, 220 mM, 225 mM, 230 mM, 240 mM, 250 mM, 255 mM, 260 mM, 270 mM, 271 mM, 272 mM, 273 mM, 274 mM, 275 mM, 280 mM, 290 mM, 300 mM, 350 mM, 400 mM, or 450 mM. In some embodiments, the concentration of L-threonine in a formulation described herein is from about 2 to about 10 mM, from about 5 to about 10 mM, from about 8 to about 12 mM, from about 8 to about 15 mM, from about 10 to about 20 mM, from about 15 to about 30 mM, from about 20 to about 30 mM, from about 25 to about 40 mM, from about 40 to about 50 mM, from about 40 to about 75 mM, from about 50 to about 100 mM, from about 75 to about 100 mM, from about 100 to about 110 mM, from 100 to 115 mM, from 110 to 120 mM, from 115 to 130 mM, from 120 to 130 mM, from 125 to 140 mM, from 140 to 150 mM, from 140 to 175 mM, from 150 to 200 mM, from 175 to 200 mM, from 200 to 215 mM, from 210 to 220 mM, from 215 to 230 mM, from 220 to 230 mM, from 225 to 240 mM, from 240 to 250 mM, from 240 to 275 mM, from 250 to 300 mM, from 250 mM to 280 mM, from 250 mM to 271 mM, from 270 to 300 mM, from 250 mM to 400 mM. In some embodiments, a high concentration of L-threonine is used, such as greater than 100 mM L-threonine, including between 100 mM and 300 mM, 100 mM and 271 mM, 150 mM and 300 mM, 150 mM and 271 mM, 200 mM and 300 mM, 200 mM and 271 mM, 225 mM and 275 mM, 225 mM and 271 mM, 250 mM and 275 mM, 250 mM and 271 mM, including 100 mM, 125 mM, 150 mM, 175 mM, 200 mM, 225 mM, 250 mM, 251 mM, 252 mM, 253 mM, 254 mM, 255 mM, 256 mM, 257 mM, 258 mM, 259 mM, 260 mM, 261 mM, 262 mM, 263 mM, 264 mM, 265 mM, 266 mM, 267 mM, 268 mM, 269 mM, 270 mM, 271 mM, 272 mM, 273 mM, 274 mM, 275 mM, 276 mM, 277 mM, 278 mM, 279 mM, 280 mM, 281 mM, 282 mM, 283 mM, 284 mM, 285 mM, 286 mM, 289 mM, 290 mM, 291 mM, 292 mM, 293 mM, 294 mM, 295 mM, 296 mM, 297 mM, 298 mM, 299 mM or 300 mM of L-threonine.

In some embodiments, the concentration of an amino acid in a formulation described herein is 5 mM. In some embodiments, the concentration of an amino acid in a formulation described herein is 10 mM, such as 10 mM L-glutamic acid. In some embodiments, the concentration of an amino acid in a formulation described herein is 30 mM. In some embodiments, the concentration of an amino acid in a formulation described herein is 65 mM. In some embodiments, the concentration of an amino acid in a formulation described herein is 100 mM. In some embodiments, the concentration of an amino acid in a formulation described herein is 250 mM, such as 250 mM L-threonine. In some embodiments, the concentration of an amino acid in a formulation described herein is 271 mM, such as 271 mM L-threonine. In some embodiments, the concentration of amino acid is 10 mM L-glutamic acid and 250 mM L-threonine, wherein the formulation also includes polysorbate, such as 0.02% w/v PS80. In some embodiments, the concentration of amino acid is 10 mM L-glutamic acid and 271 mM L-threonine, wherein the formulation also includes polysorbate, such as 0.02% w/v PS80. In some embodiments, the concentration of amino acid is 10 to 30 mM L-glutamic acid, such as 10 mM, 15 mM, 20 mM, 25 mM or 30 mM glutamic acid and 200 to 300 mM L-threonine, such as 200 mM, 201 mM, 202 mM, 203 mM, 204 mM, 205 mM, 206 mM, 207 mM, 208 mM, 209 mM, 210 mM, 211 mM, 212 mM, 213 mM, 214 mM, 215 mM, 216 mM, 217 mM, 218 mM, 219 mM, 220 mM, 221 mM, 222 mM, 223 mM, 224 mM, 225 mM, 226 mM, 227 mM, 228 mM, 229 mM, 230 mM, 231 mM, 232 mM, 233 mM, 234 mM, 235 mM, 236 mM, 237 mM, 238 mM, 239 mM, 240 mM, 241 mM, 242 mM, 243 mM, 244 mM, 245 mM, 246 mM, 247 mM, 248 mM, 249 mM, 250 mM, 251 mM, 252 mM, 253 mM, 254 mM, 255 mM, 256 mM, 257 mM, 258 mM, 259 mM, 260 mM, 261 mM, 262 mM, 263 mM, 264 mM, 265 mM, 266 mM, 267 mM, 268 mM, 269 mM, 270 mM, 271 mM, 272 mM, 273 mM, 274 mM, 275 mM, 276 mM, 277 mM, 278 mM, 279 mM, 280 mM, 281 mM, 282 mM, 283 mM, 284 mM, 285 mM, 286 mM, 289 mM, 290 mM, 291 mM, 292 mM, 293 mM, 294 mM, 295 mM, 296 mM, 297 mM, 298 mM, 299 mM or 300 mM of L-threonine wherein the formulation also includes polysorbate, such as 0.02% w/v PS80.

In some embodiments, one or more (e.g., two, three, or four) amino acids are included in a formulation described herein. In some embodiments, an amino acid in a formulation described herein is selected from the group comprising: L-glutamic acid, valine, L-threonine, proline, and any combination thereof. In some embodiments, the formulation comprises L-threonine and L-glutamic acid.

In some embodiments, the concentration of a disaccharide in a formulation described herein is from about 0.5% w/v to about 50% w/v. In some embodiments, the concentration of a disaccharide in a formulation described herein is about 0.5% w/v, about 1% w/v, about 2% w/v, about 3% w/v, about 4% w/v, about 5% w/v, about 6% w/v, about 7% w/v, about 8% w/v, about 9% w/v, about 10% w/v, about 12% w/v, about 15% w/v, about 20% w/v, about 25% w/v, about 30% w/v, about 40% w/v, or about 50% w/v. In some embodiments, the concentration of a disaccharide in a formulation described herein is from about 0.5% w/v to about 10% w/v, from about 0.50% w/v to about 50% w/v, from about 10% w/v to about 10% w/v, from about 5% w/v to about 8% w/v, from about 5% w/v to about 10% w/v, from about 10% w/v to about 12% w/v, from about 10% w/v to about 15% w/v, from about 10% w/v to about 20% w/v, from about 15% w/v to about 25% w/v, from about 20% w/v to about 30% w/v, from about 25% w/v to about 40% w/v, from about 20% w/v to about 50% w/v, or from about 40% w/v to about 50% w/v. In some embodiments, the concentration of a disaccharide in a formulation described herein is about 1% w/v. In some embodiments, the concentration of a disaccharide in a formulation described herein is about 7% w/v. In some embodiments, the concentration of a disaccharide in a formulation described herein is about 10% w/v. In some embodiments, a disaccharide in a formulation described herein is trehalose or sucrose.

In some embodiments, the concentration of a polyol in a formulation described herein is from about 0.5% w/v to about 20% w/v. In some embodiments, the concentration of a polyol in a formulation described herein is about 0.5% w/v, about 1% w/v, about 1.5% w/v, about 2% w/v, about 3% w/v, about 4% w/v, about 5% w/v, about 6% w/v, about 7% w/v, about 8% w/v, about 9% w/v, about 10% w/v, about 11% w/v, about 12% w/v, about 13% w/v, about 14% w/v, about 15% w/v, about 16% w/v, about 17% w/v, about 18% w/v, about 19% w/v, or about 20% w/v. In some embodiments, the concentration of a polyol in a formulation described herein is from about 0.5% w/v to about 1% w/v, from about 0.5% w/v to about 2% w/v, from about 1% w/v to about 2% w/v, from about 1% w/v to about 2.5% w/v, from about 2% w/v to about 3% w/v, from about 2% w/v to about 5% w/v, from about 5% w/v to about 10% w/v, from about 8% w/v to about 12% w/v, from about 10% w/v to about 15% w/v, from about 12% w/v to about 15% w/v, from about 12% w/v to about 20% w/v, from about 15% w/v to about 20% w/v, or from about 17% w/v to about 20% w/v. In some embodiments, the concentration of a polyol in a formulation described herein is about 1% w/v. In some embodiments, the concentration of a polyol in a formulation described herein is about 1.6% w/v. In some embodiments, the concentration of a polyol in a formulation described herein is about 2% w/v. In some embodiments, the concentration of a polyol in a formulation described herein is about 2.5% w/v. In some embodiments, one or more (e.g., two, three, or four) polyols are included in a formulation described herein. In some embodiments, a polyol in a formulation described herein is selected from the group comprising: glycerol, sorbitol, mannitol, and any combination thereof.

In some embodiments, the formulation does not comprise a buffer. In other embodiments, the formulation comprises a buffer. In some embodiments, the concentration of a buffer in a formulation described herein is from about 1 mM to about 300 mM. In some embodiments, the concentration of a buffer in a formulation described herein is about 1 mM, about 2 mM, about 3 mM, about 4 mM, about 5 mM, about 10 mM, about 15 mM, about 20 mM, about 25 mM, about 30 mM, about 40 mM, about 50 mM, about 60 mM, about 70 mM, about 75 mM, about 80 mM, about 90 mM, about 100 mM, about 110 mM, about 115 mM, about 120 mM, about 125 mM, about 130 mM, about 140 mM, about 150 mM, about 160 mM, about 170 mM, about 175 mM, about 180 mM, about 190 mM, about 200 mM, about 210 mM, about 215 mM, about 220 mM, about 225 mM, about 230 mM, about 240 mM, about 250 mM, about 260 mM, about 270 mM, about 275 mM, about 280 mM, about 290 mM, or about 300 mM. In some embodiments, the concentration of a buffer in a formulation described herein is from about 1 to about 5 mM, from about 1 to about 10 mM, from about 2 to about 10 mM, from about 5 to about 10 mM, from about 8 to about 12 mM, from about 8 to about 15 mM, from about 10 to about 20 mM, from about 15 to about 30 mM, from about 20 to about 30 mM, from about 25 to about 40 mM, from about 40 to about 50 mM, from about 40 to about 75 mM, from about 50 to about 100 mM, from about 75 to about 100 mM, from about 100 to about 110 mM, from about 100 to about 115 mM, from about 110 to about 120 mM, from about 115 to about 130 mM, from about 120 to about 130 mM, from about 125 to about 140 mM, from about 140 to about 150 mM, from about 140 to about 175 mM, from about 150 to about 200 mM, from about 175 to about 200 mM, from about 200 to about 215 mM, from about 210 to about 220 mM, from about 215 to about 230 mM, from about 220 to about 230 mM, from about 225 to about 240 mM, from about 240 to about 250 mM, from about 240 to about 275 mM, from about 250 to about 300 mM, or from about 270 to about 300 mM.

In some embodiments, the concentration of a buffer in a formulation described herein is 2 mM to 20 mM. In some embodiments, the concentration of a buffer in a formulation described herein is 5 mM. In some embodiments, the concentration of a buffer in a formulation described herein is 10 mM. In some embodiments, the concentration of a buffer in a formulation described herein is 20 mM.

In some embodiments, a formulation does not include a buffer or is at a pH in which the excipients do not have strong buffering capacity, such as L-glutamic acid in a formulation at pH 5.5. In some embodiments, a formulation includes two or more excipients acting as stabilizers and not as buffering agents. In some embodiments, the formulation is self-buffering. In some embodiments, the formulation has self-buffering capabilities and includes at least one amino acid that does not have strong buffering capacity at pH 5.5.

In some embodiments, the formulation comprises about 2.5% w/v glycerol and about 0.2% w/v PS80, such as 2.5% w/v glycerol and 0.2% w/v PS80. In some embodiments, the formulation comprises about 10% w/v trehalose and about 0.2% w/v PS80, such as 10% w/v trehalose and 0.02% or 0.2% w/v PS80. In some embodiments, the formulation comprises about 10 mM L-glutamic acid, about 2.5% w/v glycerol and about 0.2% w/v PS80, such as 10 mM L-glutamic acid, 2.5% w/v glycerol and 0.02% or 0.2 w/v PS80. In some embodiments, the formulation comprises about 10 mM L-glutamic acid, about 271 mM valine and about 0.2% w/v PS80. In some embodiments, the formulation comprises 10 mM L-glutamic acid, 271 mM valine and 0.02% w/v PS80. In some embodiments, the formulation comprises about 10 mM L-glutamic acid, about 65 mM valine, about 2% w/v glycerol and about 0.2% w/v PS80. In some embodiments, the formulation comprises 10 mM L-glutamic acid, 65 mM valine, 2% w/v glycerol and 0.2% w/v PS80. In some embodiments, the formulation comprises 10 mM L-glutamic acid, 65 mM valine, 2% w/v glycerol and 0.02% w/v PS80. In some embodiments, the formulation comprises about 10 mM L-glutamic acid, about 5 mM valine, about 2.5% w/v glycerol and about 0.2% w/v PS80. In some embodiments, the formulation comprises about 10 mM L-glutamic acid, about 271 mM L-threonine and about 0.2% w/v PS80. In some embodiments, the formulation comprises 10 mM L-glutamic acid, 271 mM L-threonine and 0.2% w/v PS80. In some embodiments, the formulation comprises 10 mM L-glutamic acid, 271 mM L-threonine and 0.02% w/v PS80. In some embodiments, the formulation comprises about 10 mM L-glutamic acid, about 30 mM L-threonine, about 2% w/v glycerol and about 0.2% w/v PS80. In some embodiments, the formulation comprises 10 mM L-glutamic acid, 30 mM L-threonine, 2% w/v glycerol and 0.2% w/v PS80 or 0.02% w/v PS80. In some embodiments, the formulation comprises about 10 mM L-glutamic acid, about 10 mM L-threonine, about 2.5% w/v glycerol and about 0.2% w/v PS80. In some embodiments, the formulation comprises 10 mM L-glutamic acid, 10 mM L-threonine, 2.5% w/v glycerol and 0.2% w/v PS80 or 0.2% w/v PS80. In some embodiments, the formulation comprises about 10 mM L-glutamic acid, about 100 mM proline, about 1.6% w/v glycerol and about 0.2% w/v PS80. In some embodiments, the formulation comprises about 100 mM proline, about 1.6% w/v glycerol and about 0.2% w/v PS80. In some embodiments, the formulation comprises about 10 mM L-glutamic acid, about 10% w/v trehalose and about 0.2% w/v PS80. In some embodiments, the formulation comprises about 10 mM L-glutamic acid, about 1% w/v trehalose, about 2% w/v glycerol and about 0.2% w/v PS80. In some embodiments, the formulation comprises about 10 mM L-glutamic acid, about 1% w/v sorbitol, about 2% w/v glycerol and about 0.2% w/v PS80. In some embodiments, the formulation comprises about 10 mM L-glutamic acid, about 1% w/v mannitol, about 2% w/v glycerol and about 0.2% w/v PS80. In some embodiments, the formulation comprises about 2 mM acetic acid, about 10 mM L-glutamic acid, about 2.5% w/v glycerol and about 0.2% w/v PS80 or about 0.02% w/v PS80. In some embodiments, the formulation comprises about 10 mM lactic acid, about 2.5% w/v glycerol and about 0.2% w/v PS80 or about 0.02% w/v PS80. In some embodiments, the formulation comprises about 10 mM lactic acid, about 271 mM L-threonine and about 0.2% w/v PS80 or about 0.02% w/v PS80.

In some embodiments, the one or more disclosed formulations include an antioxidant. Exemplary antioxidants include amino acids, such as methionine, L-cysteine, L-carnitine, or a mixture thereof; vitamins, such as vitamin A, vitamin C, vitamin E, or a mixture thereof, coenzyme, such as coenzyme Q10; and/or glutathione, methylsulfonyl sulfate, or a mixture thereof. In some embodiments, the one or more disclosed formulations do not include an antioxidant.

In some embodiments, the one or more disclosed formulations (e.g., formulation including threonine and/or glutamic acid, such as 25 mg/mL pembrolizumab, 10 mM L-glutamic acid, 250 or 271 mM L-threonine, and 0.02% (w/v) PS80, pH 5.5 or formulation including 50 mg/mL pembrolizumab, 10 mM L-glutamic acid, 250 or 271 mM L-threonine, and 0.02% (w/v) PS80, pH 5.5) that support a long expiry product (such as at least 2 years, including 2 years, 3 years, 4 years, 5 years or more, as liquid state, pH 5.5 at 2-8 degrees Celsius in the absence of a disaccharide-based stabilizer and/or a buffer. The disclosed formulations are stable and can be stored at 2 to 8 degrees Celsius for extended periods of time, including at least 6 months, at least 7 months, at least 8 months, at least 9 months, at least 10 months, at least 11 months, at least 12 months, at least 18 months, at least 24 months, at least 36 months, at least 48 months, at least 60 months, such as about 12 months, about 18 months, about 24 months, about 30 months, about 36 months, about 48 months, about 60 months, including 6-36 months, 6-12 months, 6-9 months, 9-12 months, 12-18 months, 12-24 months, 12-18 months, 18-24 months, 12-36 months, 24 to 36 months, such as 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 36 months, or longer.

In some embodiments, the one or more disclosed formulations has 0.1% or less than 0.1% change in total amount of HMW species in liquid state at 5° C. at 104 weeks (2 years) or up to 104 weeks compared to HMW species at zero weeks.

In some embodiments, the one or more disclosed formulations has 0.08% or less than 0.08% change in total amount of HMW species in liquid state at 5° C. at 72 weeks compared to HMW species at zero weeks.

In some embodiments, the one or more disclosed formulations has 0.07% or less than 0.07% change in total amount of HMW species in liquid state at 5° C. at 36 weeks compared to HMW species at zero weeks.

In some embodiments the one or more disclosed formulations has 0.05% or less than 0.05% change in total amount of HMW species in liquid state at 5° C. at 24 weeks compared to HMW species at zero weeks.

In some embodiments the one or more disclosed formulations has 0.03% or less than 0.03% change in total amount of HMW species in liquid state at 5° C. at 12 weeks compared to HMW species at zero weeks.

In some embodiments the one or more disclosed formulations has 0.03% or less than 0.03% change in total amount of HMW species in liquid state at 5° C. at 8 weeks compared to HMW species at zero weeks.

In some embodiments the one or more disclosed formulations has 0.02% or less than 0.02% change in total amount of HMW species in liquid state at 5° C. at 4 weeks compared to HMW species at zero weeks.

In some embodiments, the one or more disclosed formulations has 0.2% or less total amount of HMW species in liquid state at a given time period at 5° C., including at 4 weeks, 8 weeks, 12 weeks, 24 weeks, 36 weeks, 72 weeks or 104 weeks.

In some embodiments, the one or more disclosed formulations has 0.1% or less total amount of HMW species in liquid state at a given time period at 25° C., including at 2 weeks or 4 weeks.

In some embodiments, the one or more disclosed formulations has 0.4% or less total amount of HMW species in liquid state at a given time period at 25° C., including at 4 weeks, 8 weeks, 12 weeks, or 24 weeks.

In some embodiments, the disclosure provides a method of treatment comprising administering a pharmaceutical formulation (or a lyophilized formulation thereof once reconstituted, e.g., with sterile water for injection) as described herein to a subject having or at risk of developing a disease or condition. In some embodiments, the method further comprises administering to the subject a second therapeutic composition. In some embodiments, the subject is a mammal. In some embodiments, the mammal is a human. In some embodiments, the disclosure provides for compositions comprising any of the pharmaceutical formulations (or lyophilized formulations thereof) as described herein for the treatment of a disease or disorder. In some embodiments, the disclosure provides for use of a pharmaceutical formulation (or a lyophilized formulation thereof) as described herein for the preparation of a medicament for the treatment of a disease or condition.

Exemplary diseases or conditions include, but are not limited to, cancer, melanoma, renal cancer, non-small-cell lung cancer, bladder cancer, head and neck cancer, anaplastic thyroid cancer, and infectious disease. Infectious diseases include, but are not limited to, malaria, acquired immune deficiency (AIDS), cytomegalovirus infection and influenza. Exemplary diseases or conditions also include, but are not limited to, melanoma, lung cancer including non-small cell lung cancer (NSCLC), head and neck cancer including head and neck squamous cell cancer (HNSCC), Hodgkin's lymphoma, including classical Hodgkin Lymphoma (cHL), B-cell lymphoma, including mediastinal large B-cell lymphoma (PMBCL), urothelial carcinoma, renal cell carcinoma, gastric cancer, microsatellite instability-high cancer (MSI-H), mismatch repair deficient; (dMMR), including MSI-H and dMMR colorectal cancer, can be used to treat solid cancer, cervical cancer, liver cancer, Merkel cell carcinoma (MCC), esophageal cancer, hepatocellular carcinoma (HCC), endometrial carcinoma, tumor mutational burden-high (TMB-H) cancer, cutaneous squamous cell carcinoma (cSCC), triple-negative breast cancer (TNBC), and the like.

In some embodiments, the disclosed pharmaceutical formulation further includes an endoglycosidase hydrolase enzyme. In some embodiments, the endoglycosidase hydrolase enzyme is one disclosed in US20220233693A1, US20220089738A1 or WO2020197230A1, such as a recombinant human hyaluronidase disclosed in US20220233693A1, US20220089738A1 or WO2020197230A1 each of which is hereby incorporated by reference in its entirety. In some aspects, the pharmaceutical composition comprises at least about 5 U to at least about 100,000 U of the endoglycosidase hydrolase enzyme. In some aspects, the pharmaceutical composition comprises at least about 5 U, at least about 10 U, at least about 20 U, at least about 30 U, at least about 40 U, at least about 50 U, at least about 75 U, at least about 100 U, at least about 200 U, at least about 300 U, at least about 400 U, at least about 500 U, at least about 750 U, at least about 1000 U, at least about 2000 U, at least about 3000 U, at least about 4000 U, at least about 5000 U, at least about 6000 U, at least about 7000 U, at least about 8000 U, at least about 9000 U, at least about 10,000 U, at least about 20,000 U, at least about 30,000 U, at least about 40,000 U, at least about 50,000 U, at least about 60,000 U, at least about 70,000 U, at least about 80,000 U, at least about 90,000 U, or at least about 100,000 U of the endoglycosidase hydrolase enzyme. In some aspects, the pharmaceutical composition comprises about 20,000 U of the endoglycosidase hydrolase enzyme. In some aspects, the pharmaceutical composition comprises at least about 500 U/mL to at least about 5000 U/mL of the endoglycosidase hydrolase enzyme. In some aspects, the pharmaceutical composition comprises at least about 1500 U/mL, at least about 1600 U/mL, at least about 1700 U/mL, at least about 1800 U/mL, at least about 1900 U/mL, at least about 2000 U/mL, at least about 2100 U/mL, at least about 2200 U/mL, at least about 2300 U/mL, at least about 2400 μM, at least about 2500 μM, at least about 3000 μM, at least about 3500 μM, at least about 4000 μM, at least about 4500 U/mL, or at least about 5000 U/mL of the endoglycosidase hydrolase enzyme. In some embodiments, the pharmaceutical composition comprises about 2000 U/mL of the endoglycosidase hydrolase enzyme.

In some embodiments, pharmaceutical formulations (or lyophilized formulations thereof) as described herein may be administered subcutaneously, intravenously, parenterally, intradermally, intramuscularly, and/or intraperitoneally using standard techniques. For example, a stable pharmaceutical formulation as described herein may be prepared to be subcutaneously administered using a pre-filled syringe. Specifically, any of the formulations described herein may be administered once every week or 6 to 8 days or 7 to 10 days, or every other week or every two weeks or 12 to 16 days or 7 to 14 days or 13 to 15 days, or every three weeks or 19 to 23 days, or every month or 26 to 30 days or 29 to 31 days, or every five weeks or 33 to 34 days, or every six weeks or 40 to 44 days, or every seven weeks or 47 to 51 days, or every two months or 54 to 58 days subcutaneously, intravenously, parenterally, intradermally, intramuscularly, and/or intraperitoneally at a therapeutically effective dosage and in the formulations described herein for an indefinite period of time for the treatment of the diseases and conditions described above.

Administration and dosage regimens of stable pharmaceutical formulations (or lyophilized formulations thereof) as described herein can be adjusted to provide an effective amount for an optimum therapeutic response. For example, a single bolus can be administered, two or more divided doses can be administered over time or the dose can be proportionally reduced or increased as indicated by the exigencies of the therapeutic situation. For example, a unit dose can be administered over two consecutive days every two weeks. Unit dosing refers to a physically discrete amount of an anti-PD-1 antibody (e.g., pembrolizumab), suited as unitary dosages for the patients to be treated; each unit contains a predetermined quantity of active biopharmaceutical calculated to produce a desired therapeutic effect.

The dosing regimen of stable pharmaceutical formulations (or reconstituted lyophilized formulations thereof) as described herein may comprise administering a dose given on Day one, followed by the administration of the same dose every other week. The dosing regimen of stable pharmaceutical formulations (or lyophilized formulations thereof) as described herein may comprise administering an initial dose given on day one or split over two consecutive days, followed by the administration of the same or a reduced dose two weeks later (Day 15), e.g., the initial dose reduced by half. The dosing regimen may further comprise administration of the same or further reduced dose two weeks later (Day 29); e.g., a dose that is a fourth of the initial dose which will be continued as a maintenance dose every two weeks.

For example, the dosage regimen for the treatment of at least melanoma, non-small cell lung cancer (NSCLC), head and neck squamous cell cancer (HNSCC), classical Hodgkin lymphoma (cHL) or primary mediastinal large B-cell lymphoma (PMBCL), urothelial carcinoma, renal cell carcinoma, microsatellite instability-high (MSI-H) or mismatch repair deficient (dMMR) cancer, MSI-H or dMMR colorectal cancer (CRC), gastric cancer, esophageal cancer, cervical cancer, hepatocellular carcinoma (HCC), liver cancer, merkel cell carcinoma (MCC), endometrial carcinoma, tumor mutational burden-high (TMB-H) cancer, cutaneous squamous cell carcinoma (cSCC), and/or triple-negative breast cancer (TNBC) can be 200 mg every 3 weeks or 400 mg every 6 weeks. Moreover, for pediatrics, the dosage regimen for at least melanoma, cHL or PMBCL, MSI-H or dMMR cancer, MCC, and/or TMB-H cancer 2 mg/kg (up to 200 mg) every 3 weeks for pediatrics. The dosage regimen for the treatment of renal cell carcinoma (RCC) can be 200 mg every 3 weeks or 40 mg every 6 weeks as a single agent in the adjuvant setting, or in the advanced setting with either 5 mg axitinib orally twice daily or 20 mg lenvatinib orally once daily. The dosage regimen for endometrial carcinoma can be 200 mg every 3 weeks or 40 mg every 6 weeks with 20 mg lenvatinib orally once daily.

In some embodiments, the disclosure provides for a method of preparing a pharmaceutical formulation as described herein, comprising combining an aqueous solution comprising one or more excipients and a therapeutically effective amount of an anti-PD-1 antibody (e.g., pembrolizumab), using techniques standard in the art. The invention further provides for a method of preparing the stable lyophilized pharmaceutical formulations as described herein, comprising lyophilizing an aqueous pharmaceutical formulation comprising one or more excipients and a therapeutically effective amount of an anti-PD-1 antibody (e.g., pembrolizumab), using techniques standard in the art.

The foregoing detailed description is not intended to define every aspect of the disclosure, and other features and advantages of the present disclosure will be apparent to those skilled in the art. The present disclosure is intended to be related as a unified document, and it should be understood that all combinations of features described herein are contemplated, even if the combination of features are not found together in the same sentence, paragraph, or section of this disclosure. In addition, the disclosure includes, as an additional aspect, all embodiments of the invention narrower in scope in any way than the variations specifically mentioned above. With respect to aspects of the disclosure described or claimed with “a” or “an,” it should be understood that these terms mean “one or more” unless context unambiguously requires a more restricted meaning. With respect to elements described as one or more within a set, it should be understood that all combinations within the set are contemplated. If aspects of the disclosure are described as “comprising” a feature, embodiments also are contemplated “consisting of” or “consisting essentially of” the feature. Additional features and variations of the disclosure will be apparent to those skilled in the art from the entirety of this application, and all such features are intended as aspects of the disclosure.

The present disclosure will be more readily understood by reference to the following examples, which are provided by way of illustration and are not intended to be limiting.

### EXAMPLES

The following Examples describe exemplary formulations of the present disclosure.

### Example 1: Aggregation Rate Assessment in Exemplary Pembrolizumab Formulations

A series of excipients including different buffers and stabilizers were used to produce new pembrolizumab formulations. Exemplary formulations comprising 25 mg/mL of a pembrolizumab biosimilar and different excipients in different amounts are listed in Table 1A. The stability of the exemplary formulations was tested and compared to that of a Reference Product (RP, Keytruda®) formulation (25 mg/mL pembrolizumab, 10 mM histidine, 7% sucrose, 0.02% Polysorbate 80 (w/v)) using high-throughput screening and long-term stability studies.

TABLE 1A
Exemplary formulations including 25 mg/ml pembrolizumab
biosimilar, PS80 0.02% (w/v), pH 5.5.

| Formulation Name | Formulation Composition |
|---|---|
| Glu-Glyc | 10 mM L-glutamic acid, 2.5% w/v glycerol |
| Glyc | 2.5% w/v glycerol |
| Glu-high Val | 10 mM L-glutamic acid, 271 mM valine |
| Glu-med Val-Glyc | 10 mM L-glutamic acid, 65 mM valine, 2% w/v glycerol |
| Glu-low Val-Glyc | 10 mM L-glutamic acid, 5 mM valine, 2.5% w/v glycerol |
| Glu-high Thr | 10 mM L-glutamic acid, 271 mM L-threonine |
| Glu-med Thr-Glyc | 10 mM L-glutamic acid, 30 mM L-threonine, 2% w/v glycerol |
| Glu-low Thr-Glyc | 10 mM L-glutamic acid, 10 mM L-threonine, 2.5% w/v glycerol |
| Lac-Glyc | 10 mM lactic acid, 2.5% w/v glycerol |
| Lac-high Thr | 10 mM lactic acid, 271 mM L-threonine |
| Glu-Pro-Glyc | 10 mM L-glutamic acid, 100 mM proline, 1.6% w/v glycerol |
| Pro-Glyc | 100 mM proline, 1.6% w/v glycerol |
| Glu-high Treh | 10 mM L-glutamic acid, 10% w/v trehalose |
| Tre | 10% w/v trehalose |
| Glu-low Treh-Glyc | 10 mM L-glutamic acid, 1% w/v trehalose, 2% w/v glycerol |
| Glu-Sor-Glyc | 10 mM L-glutamic acid, 1% w/v sorbitol, 2% w/v glycerol |
| Glu-Mann-Glyc | 10 mM L-glutamic acid, 1% w/v mannitol, 2% w/v glycerol |
| Ace-Glu-Glyc | 2 mM acetic acid, 10 mM L-glutamic acid, 2.5% w/v glycerol |
| Reference Product (RP) | 10 mM histidine, 7% w/v sucrose |

Prepared samples of the exemplary formulations were sterile filtered and aseptically filled in 6R vials. Samples were incubated at a temperature of 2-8° C. or 25° C. for up to four weeks. At each time point, the level of aggregation in each sample was evaluated using Size Exclusion-UltraHigh Performance Liquid Chromatography (SE-UHPLC), Cation Exchange chromatography (CEX), and reduced Capillary Electrophoresis (rCE).

SE-UHPLC can be performed to monitor monoclonal antibody aggregation, such as anti-PD1 antibody aggregation, including pembrolizumab formulations disclosed herein. Size exclusion chromatography (SEC) is a routine technique for the analysis of proteins. SEC columns can be used to evaluate the aggregation profile of a protein. For example, SEC can be used to determine the amount of HMW species and to monitor the change in amount of % HMW species over a selected period of time in an anti-PD-1 formulation, such as one or more of the disclosed pembrolizumab formulations. SEC can be performed by the techniques known to one of skill in the art. General teachings of how SEC can be performed to determined aggregation can be found in A. E. Hamielec, S. T, Balke, B. P. Leclair & S. L. Pearce; Ind. Eng. Chem., Prod. Res. Dev., 8,54 (1969), Z. Grubistic, R. Rempp & H. Benoit; J. Polymer Sci., Part B, 5, 753 (1967) “Polymer Handbook”, J. Brandrup, E. H. Immersut: John Wiley & Sons Publisher. Data Reduction in Multidetector Size Exclusion Chromatography, Y Brun J. Liq. Chrom & Rel. Technology, 21 (13), 1979-2015 (1998); Modern Size-Exclusion Liquid Chromatography, W. W. Yau, J. J. Kirkland, D. D. Bly John Wiley & Sons, Inc., New York, 1979, each of which is hereby incorporated by reference in its entirety.

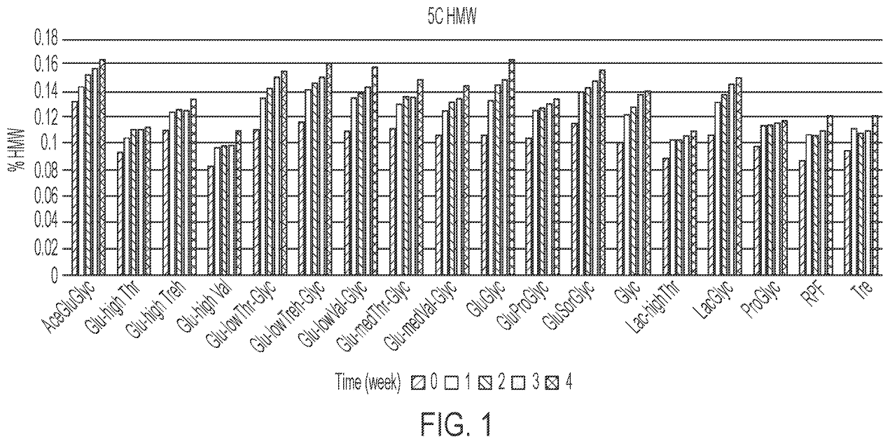

FIG. 1 shows comparable aggregation rates (<0.05% HMW difference) at 5° C. for the different formulations tested and for the RP formulation at each time point up to 4 weeks. Pembrolizumab biosimilar (25 mg/mL) formulations including PS80 0.02% (w/v) with aggregation rates that were similar to those of the RP formulation over time, include Tre (10% w/v trehalose, pH 5.5), Pro-Glyc (100 mM proline, 1.6% w/v glycerol, pH 5.5), Glu-high Val (10 mM L-glutamic acid, 271 mM valine, pH 5.5), Glu-high Treh (10 mM L-glutamic acid, 10% w/v trehalose, pH 5.5) and Glu-high Thr (10 mM L-glutamic acid, 271 mM L-threonine, pH 5.5) formulations..

Additional formulations provided in Table 1B below were evaluated; all test formulations were solution formulations at pH 5.5 containing 25 mg/mL pembrolizumab.

TABLE 1B
Exemplary formulations

| Formulation Composition | 0 weeks | 1 weeks | 2 weeks | 3 weeks | 4 weeks |
|---|---|---|---|---|---|
| 10 mM histidine, 7% w/v sucrose, 0.02% w/v | 0.5 | 2.7 | 6.1 | 6.2 | 9.8 |
| polysorbate 80 | | | | | |
| 10 mM L-glutamic acid, 100 mM asparagine, 2.5% | 0.6 | 3.3 | 5.6 | 4.7 | 9.2 |
| w/v sorbitol, 0.02% w/v polysorbate 80 | | | | | |
| 10 mM L-glutamic acid, 100 mM asparagine, 2.5% | 0.6 | 3.2 | 3.7 | 5 | 6.1 |
| w/v sorbitol, 0.1% w/v poloxmer 188 | | | | | |
| 10 mM glutamic acid, 100 mM asparagine, 11.7 nM | 0.6 | 2.8 | 4.0 | 4.2 | 2.1 |
| N-acetyl methionine, 2.5% w/v sorbitol, 0.02% w/v | | | | | |
| polysorbate 80 | | | | | |
| 10 mM glutamic acid, 2.2% w/v glycerol, 0.02% w/v | 0.6 | 3.6 | 7.1 | 6.9 | 11.9 |
| polysorbate 80 | | | | | |
| 10 mM succinate, 100 mM asparagine, 2.5% w/v | 0.6 | 3.5 | 3.6 | 6.6 | 6 |
| sorbitol, 0.02% w/v PS80 | | | | | |
| 10 mM succinate 12 mM N-acetyl methionine, 2.2% | 0.6 | 3.3 | 6.3 | 5.0 | 4.6 |
| w/v glycerol, 0.1% w/v P188 | | | | | |
| 10 mM succinate, 100 mM asparagine, 14.3 mM | 1.0 | 4.3 | NT | NT | NT |
| MgCl2, 2.5% w/v sorbitol, 0.02% PS80 | | | | | |
| 10 mM citrate, 100 mM asparagine, 2.5% w/v | 0.9 | 2.6 | 3.8 | NT | NT |
| sorbitol, 0.02% w/v PS80 | | | | | |

### Example 2: Stability of Exemplary Pembrolizumab Formulations

In order to further assess the formulation stability within a representative manufacturing setting, five exemplary formulations comprising 25 mg/mL of pembrolizumab were subjected to a manufacturing processing model. The formulations tested are listed in Table 2 and include a reference product formulation to commercially available pembrolizumab formulation (Keytruda®).

TABLE 2
Exemplary formulations including 25 mg/mL
pembrolizumab and PS80 0.02% (w/v), pH 5.5

| Formulation Name | Formulation Composition |
|---|---|
| Glu-Glyc | 10 mM L-glutamic acid, 2.5% w/v glycerol |
| Glu-high Thr | 10 mM L-glutamic acid, 271 mM L-threonine |
| Pro-Glyc | 100 mM proline, 1.6% w/v glycerol |
| Tre (or Treh or high Treh) | 10% w/v trehalose |
| Reference Product Formulation (RPF) | 10 mM histidine, 7% w/v sucrose |

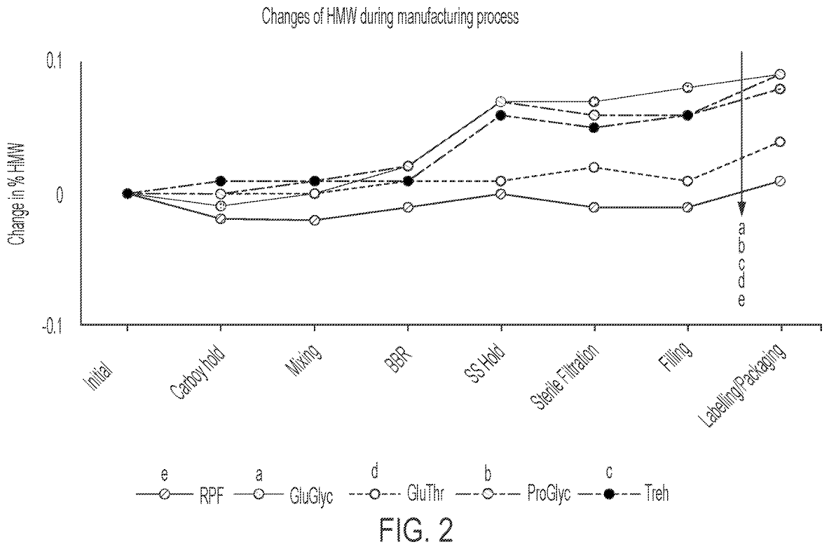

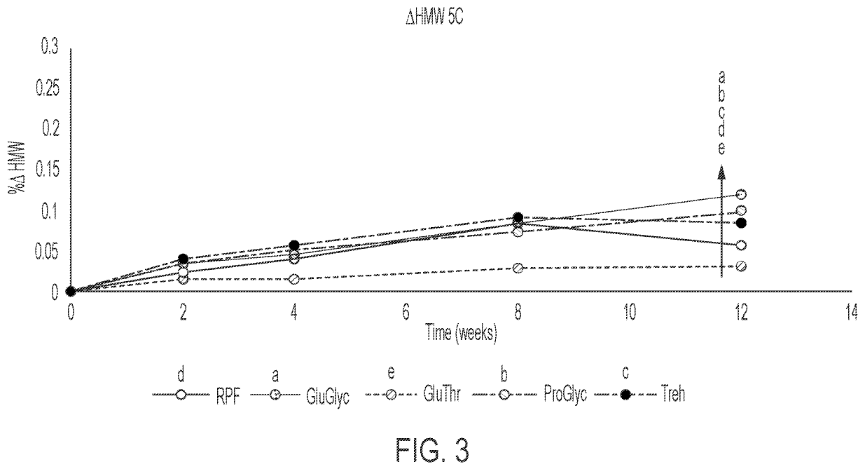

FIG. 2 shows that the manufacturing process had little impact on the level of aggregation in the tested formulations. Further stability analysis assessing the change in % HMW at 5° C. at 0, 2, 4, 8 and 12 weeks showed that all five tested formulations had comparable aggregation rates at 5° C. at the respective timepoints (FIG. 3). These results confirm that the Glu-Glyc (10 mM L-glutamic acid, 2.5% w/v glycerol), Glu-high Thr (10 mM L-glutamic acid, 271 mM L-threonine), Pro-Glyc (100 mM proline, 1.6% w/v glycerol) and Tre (10% w/v trehalose) formulations, which include no buffer, have similar stability to that of the RPF. Additional studies indicated that 25 mg/mL pembrolizumab, 0.02% (w/v) PS80, Glu-high Thr (10 mM L-glutamic acid, 250 mM L-threonine) formulations had dramatically lower % change in HMW species at all time points tested (2 weeks, 4 weeks, 8 weeks, 12 weeks and 24 weeks) as shown in Table 3 below (detection method for % HMW accurate to 0.1%).

TABLE 3
Percent Change in HMW species in Evaluated Formulations

| | 0 | 2 | 4 | 8 | 12 | 24 |
|---|---|---|---|---|---|---|
| RP | 0 | 0.02611 | 0.0410638 | 0.084597 | 0.06 | 0.091805956 |
| GluThr | 0 | 0.016629 | 0.015729 | 0.028113 | 0.03 | 0.052988154 |

### Example 3: Stability of Pembrolizumab Formulations Through Manufacturing Processing Stress and Over Time

In order to further assess the formulation stability, formulations comprising 25 mg/mL or 50 mg/mL pembrolizumab biosimilar were subjected to a worst-case manufacturing processing model. One formulation included 25 mg/mL pembrolizumab biosimilar, 10 mM L-glutamic acid, 250 mM L-threonine, and 0.02% (w/v) PS80, pH 5.5 and a second formulation included 50 mg/mL pembrolizumab biosimilar, 10 mM L-glutamic acid, 250 mM L-threonine, and 0.02% (w/v) PS80, pH 5.5. The RPF is 25 mg/mL pembrolizumab, 10 mM L-histidine, 7% (w/v) sucrose, and 0.02% (w/v) PS80, pH 5.5.

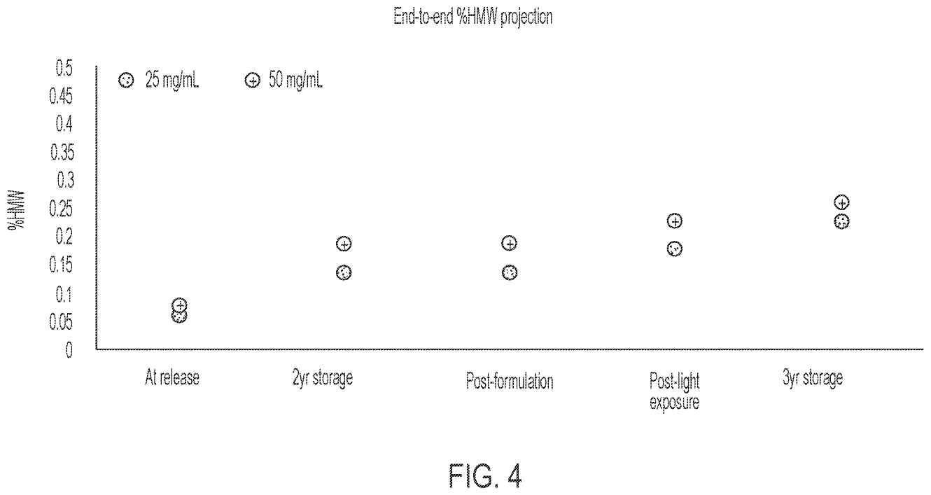

FIG. 4 shows that the anticipated worst-case lab-scale processing stresses did not impact the aggregation rates of monomer of monoclonal antibody in the tested formulation. The extended arm studies did not impact pembrolizumab biosimilar product qualities. The HMW of these formulations are predicted to remain within the quality target product profile at end of its shelf life. Stability data at recommended storage conditions (RSC) show no impact on product quality after worst-case processing stresses.

The stability analysis assessing the change in % HMW has shown that formulations post worst-case lab-scale processing steps are stable at 2-8 degrees Celsius at tested timepoints. Experimental values show that disclosed formulations can maintain stability over end-to-end storage and processing steps. Typically, a disaccharide-based stabilizer, such as sucrose or trehalose, and/or a strong buffer is needed for a stable liquid monoclonal antibody formulation. Surprisingly, disclosed herein are formulations (e.g., formulation including threonine, such has high concentrations of threonine and/or glutamic acid, such as 25 mg/mL pembrolizumab, 10 mM L-glutamic acid, 250 or 271 mM L-threonine, and 0.02% (w/v) PS80, pH 5.5 or formulation including 50 mg/mL pembrolizumab 10 mM L-glutamic acid, 250 or 271 mM L-threonine, and 0.02% (w/v) PS80, pH 5.5) that support a long expiry product (such as at least 2 years, including 2 years, 3 years, 4 years, 5 years or more, as liquid state, pH 5.5 at 2-8 degrees Celsius in the absence of a disaccharide-based stabilizer and/ a strong buffer. The disclosed formulations are stable and can be stored at 2 to 8 degrees Celsius for extended periods of time, including at least 6 months, at least 12 months, at least 18 months, at least 24 months, at least 36 months, at least 48 months, at least 60 months, such as about 12 months, about 18 months, about 24 months, about 30 months, about 36 months, about 48 months, about 60 months, including 6-36 months, 6-12 months, 6-9 months, 9-12 months, 12-18 months, 12-24 months, 12-18 months, 18-24 months, 12-36 months, 24 to 36 months, such as 6 months, 7 months, 8 months, 9 months, 10 months, 11 months, 12 months, 18 months, 24 months, 30 months, 36 months, or longer.

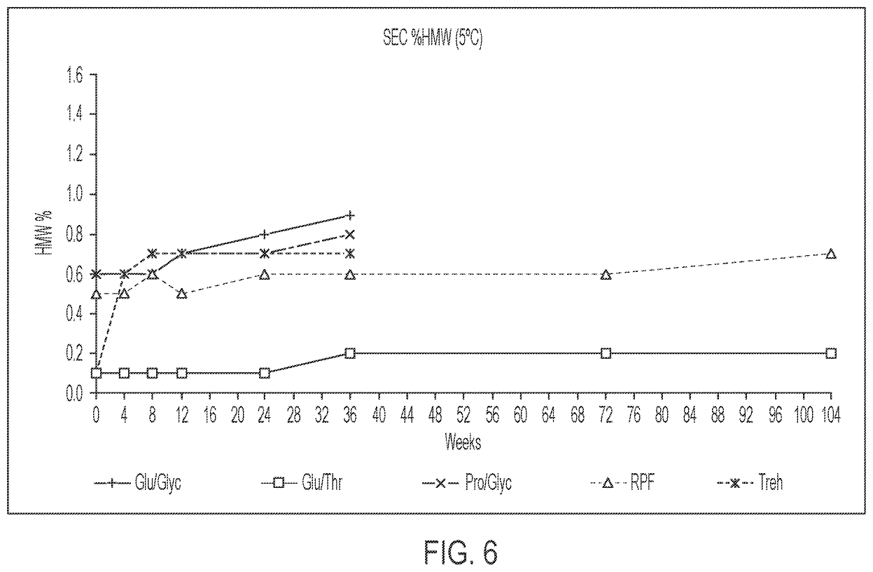

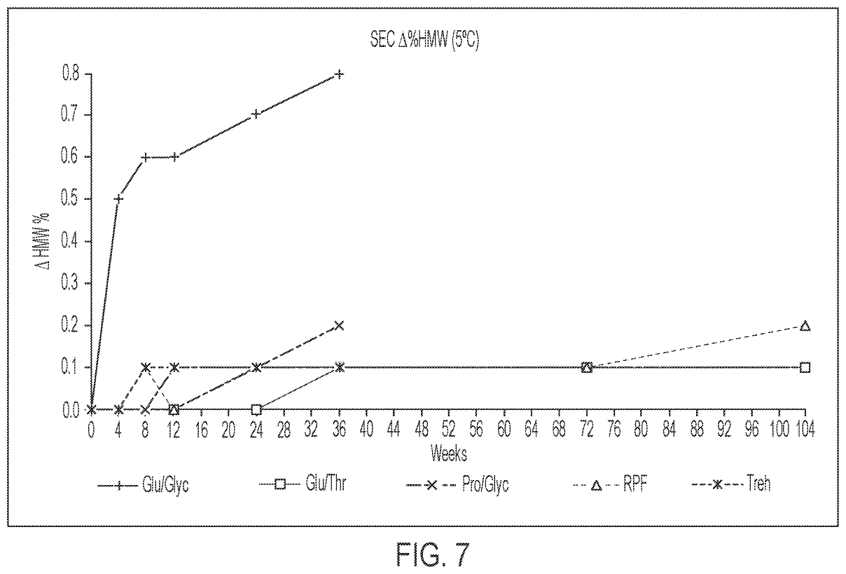

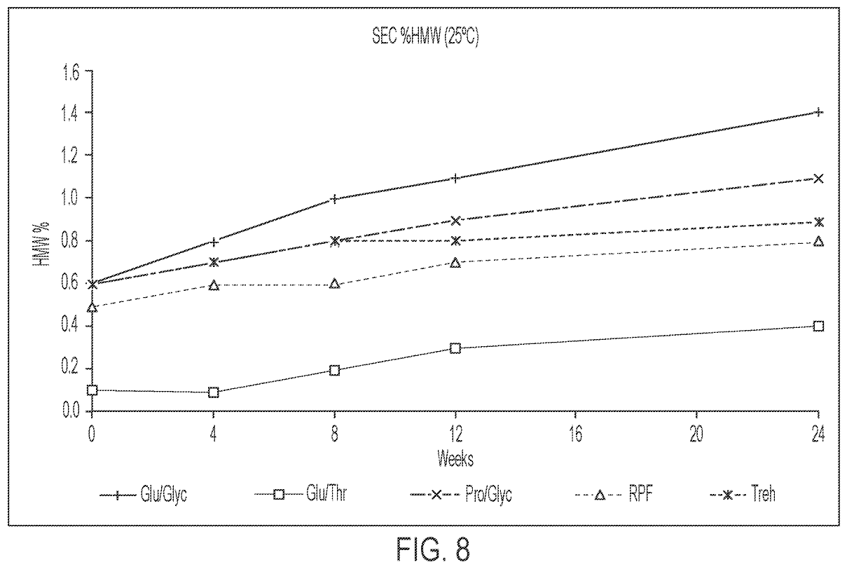

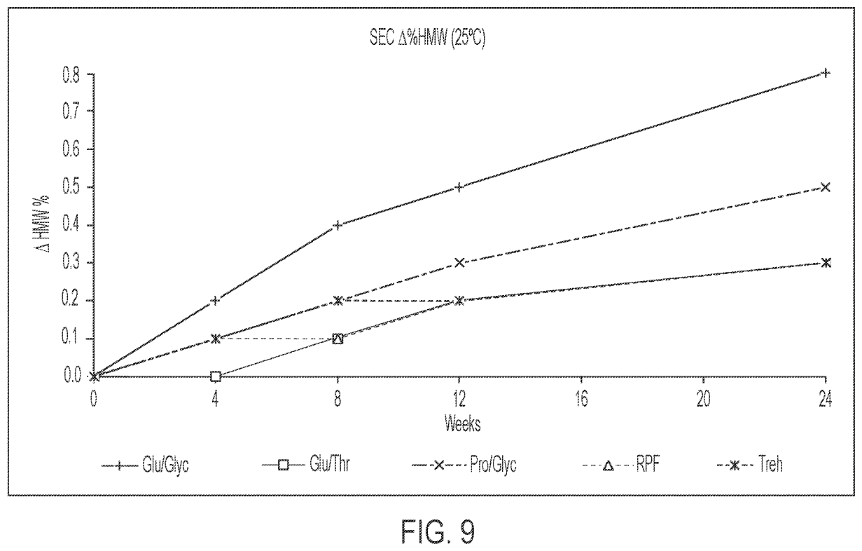

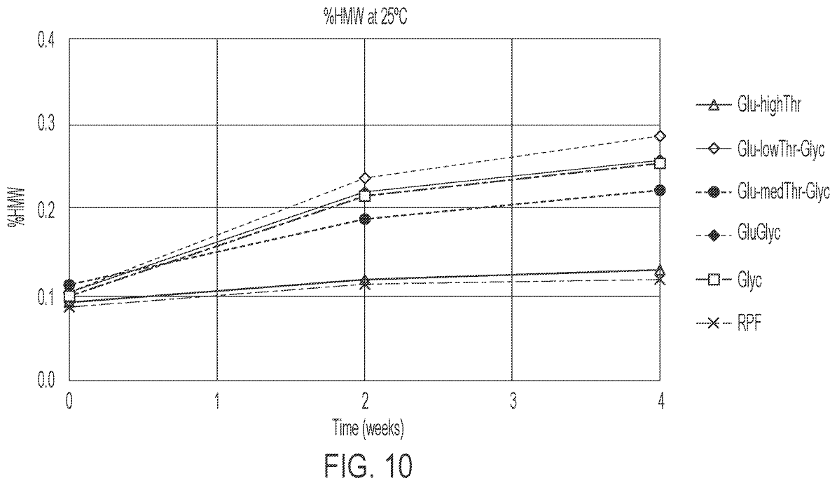

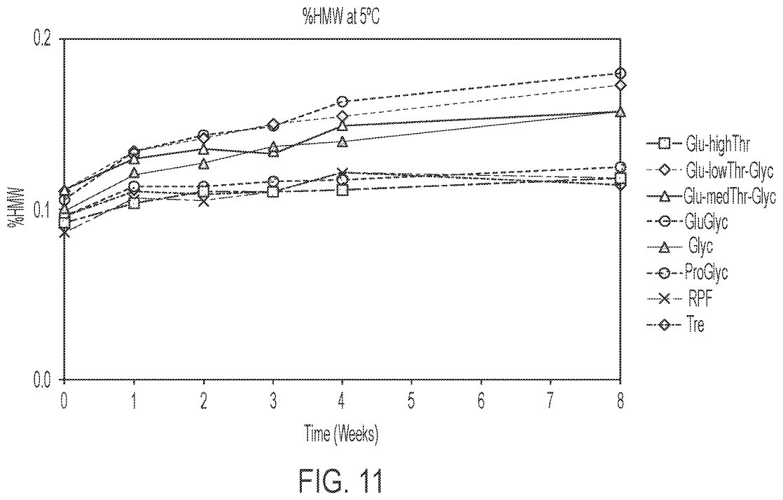

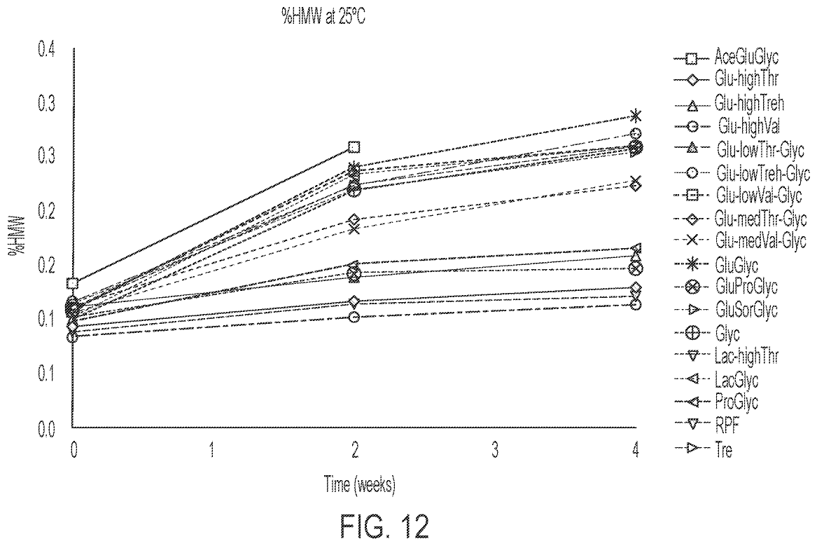

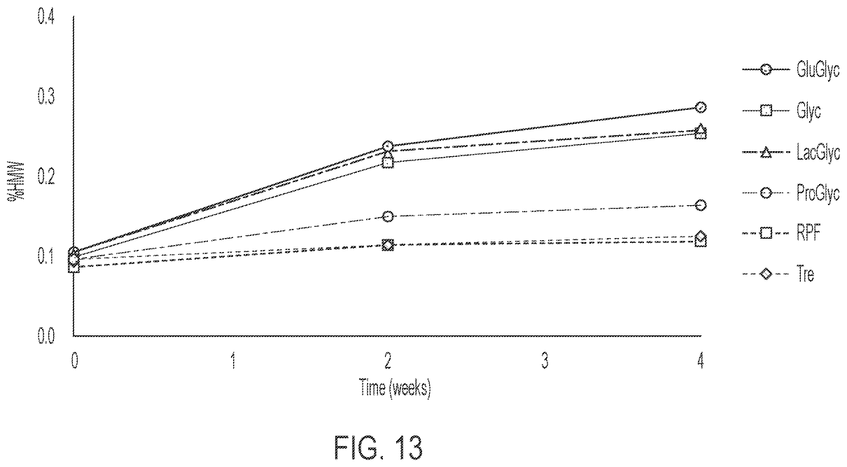

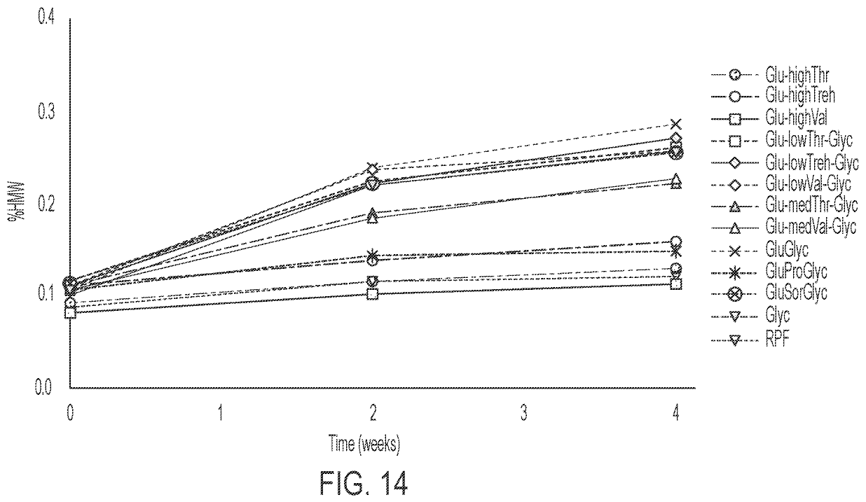

Table 4 provides amount of aggregation (% HMW) in various formulations at zero, 4, 8, 12, 24, 36, 72, or 104 weeks at 5° C. The data from Table 4 is represented in FIG. 6. Table 5 provides change in aggregation (% HMW) in various formulations at zero, 4, 8, 12, 24, 36, 72, or 104 weeks at 5° C. The data from Table 5 is represented in FIG. 7. Table 6 provides amount of aggregation (% HMW) in various formulations at zero, 4, 8, 12, or 24 weeks at 25° C. The data from Table 6 is represented in FIG. 8. Table 7 provides change in aggregation (% HMW) at zero, 4, 8, 12, or 24 weeks at 25° C. The data from Table 7 is represented in FIG. 9. Table 8 provides amount of aggregation (% HMW) in various formulations at zero, 2, or 4 weeks at 25° C. The data from Table 8 is represented in FIG. 10. Table 9 provides amount of aggregation (% HMW) in various formulations at zero, 1, 2, 3, 4, or 8 weeks at 5° C. The data from Table 9 is represented in FIG. 11. Table 10 provides amount of aggregation (% HMW) in various formulations at zero, 2, or 4 weeks at 25° C. The data from Table 10 is represented in FIG. 12. Table 11 provides amount of aggregation (% HMW) in various formulations at zero, 2, or 4 weeks at 25° C. The data from Table 11 is represented in FIG. 13. Table 12 provides amount of aggregation (% HMW) in various formulations at zero, 2, or 4 weeks at 25° C. The data from Table 12 is represented in FIG. 14.

TABLE 4
Percent HMW in Evaluated Formulations (5° C.)

| | | 0 | 4 | 8 | 12 | 24 | 36 | 72 | 104 |
|---|---|---|---|---|---|---|---|---|---|
| Formulation | Label | weeks | weeks | weeks | weeks | weeks | weeks | weeks | weeks |
| 10 mM glutamic acid, | Glu/Glyc | 0.1 | 0.6 | 0.7 | 0.7 | 0.8 | 0.9 | NT | NT |
| 2.5% w/v glycerol | | | | | | | | | |
| 10 mM glutamic acid, | Glu/Thr | 0.1 | 0.1 | 0.1 | 0.1 | 0.1 | 0.2 | 0.2 | 0.2 |
| 250 mM threonine | | | | | | | | | |
| 100 mM proline, | Pro/Glyc | 0.6 | 0.6 | 0.6 | 0.7 | 0.7 | 0.8 | NT | NT |
| 1.6% w/v glycerol | | | | | | | | | |
| 10 mM histidine, | RPF | 0.5 | 0.5 | 0.6 | 0.5 | 0.6 | 0.6 | 0.6 | 0.7 |
| 7% w/v sucrose | | | | | | | | | |
| 10% w/v trehalose | Treh | 0.6 | 0.6 | 0.7 | 0.7 | 0.7 | 0.7 | NT | NT |

All formulations contain 25 mg/mL pembrolizumab, 0.02% w/v polysorbate 80 at pH 5.5;
NT = not tested

TABLE 5
Change in Percent HMW in Evaluated Formulations (5° C.)

| | | 0 | 4 | 8 | 12 | 24 | 36 | 72 | 104 |
|---|---|---|---|---|---|---|---|---|---|
| Formulation | Label | weeks | weeks | weeks | weeks | weeks | weeks | weeks | weeks |
| 10 mM glutamic acid, | Glu/Glyc | 0.0 | 0.5 | 0.6 | 0.6 | 0.7 | 0.8 | NT | NT |
| 2.5% w/v glycerol | | | | | | | | | |
| 10 mM glutamic acid, | Glu/Thr | 0.0 | 0.0 | 0.0 | 0.0 | 0.0 | 0.1 | 0.1 | 0.1 |
| 250 mM threonine | | | | | | | | | |
| 100 mM proline, | Pro/Glyc | 0.0 | 0.0 | 0.0 | 0.1 | 0.1 | 0.2 | NT | NT |
| 1.6% w/v glycerol | | | | | | | | | |
| 10 mM histidine, | RPF | 0.0 | 0.0 | 0.1 | 0.0 | 0.1 | 0.1 | 0.1 | 0.2 |
| 7% w/v sucrose | | | | | | | | | |
| 10% w/v trehalose | Treh | 0.0 | 0.0 | 0.1 | 0.1 | 0.1 | 0.1 | NT | NT |

All formulations contain 25 mg/mL pembrolizumab, 0.02% w/v polysorbate 80 at pH 5.5;
NT = not tested

TABLE 6
Percent HMW in Evaluated Formulations (25° C.)

| | | 0 | 4 | 8 | 12 | 24 |
|---|---|---|---|---|---|---|
| Formulation | Label | weeks | weeks | weeks | weeks | weeks |
| 10 mM glutamic acid, | Glu/Glyc | 0.6 | 0.8 | 1.0 | 1.1 | 1.4 |
| 2.5% w/v glycerol | | | | | | |
| 10 mM glutamic acid, | Glu/Thr | 0.1 | 0.1 | 0.2 | 0.3 | 0.4 |
| 250 mM threonine | | | | | | |
| 100 mM proline, | Pro/Glyc | 0.6 | 0.7 | 0.8 | 0.9 | 1.1 |
| 1.6% w/v glycerol | | | | | | |
| 10 mM histidine, | RPF | 0.5 | 0.6 | 0.6 | 0.7 | 0.8 |
| 7% w/v sucrose | | | | | | |
| 10% w/v trehalose | Treh | 0.6 | 0.7 | 0.8 | 0.8 | 0.9 |

All formulations contain 25 mg/mL pembrolizumab, 0.02% w/v polysorbate 80 at pH 5.5

TABLE 7
Change in Percent HMW in Evaluated Formulations (25° C.)

| | | 0 | 4 | 8 | 12 | 24 |
|---|---|---|---|---|---|---|
| Formulation | Label | weeks | weeks | weeks | weeks | weeks |
| 10 mM glutamic acid, | Glu/Glyc | 0.0 | 0.2 | 0.4 | 0.5 | 0.8 |
| 2.5% w/v glycerol | | | | | | |
| 10 mM glutamic acid, | Glu/Thr | 0.0 | 0.0 | 0.1 | 0.2 | 0.3 |
| 250 mM threonine | | | | | | |
| 100 mM proline, | Pro/Glyc | 0.0 | 0.1 | 0.2 | 0.3 | 0.5 |
| 1.6% w/v glycerol | | | | | | |
| 10 mM histidine, | RPF | 0.0 | 0.1 | 0.1 | 0.2 | 0.3 |
| 7% w/v sucrose | | | | | | |
| 10% w/v trehalose | Treh | 0.0 | 0.1 | 0.2 | 0.2 | 0.3 |

All formulations contain 25 mg/mL pembrolizumab, 0.02% w/v polysorbate 80 at pH 5.5

TABLE 8
Percent HMW in Evaluated Formulations (25° C.)

| Formulation | Label | 0 weeks | 2 weeks | 4 weeks | Δ % HMW |
|---|---|---|---|---|---|
| 10 mM glutamic acid, | Glu-highThr | 0.1 | 0.1 | 0.1 | 0.0 |
| 271 mM threonine | | | | | |
| 10 mM glutamic acid, | Glu-lowThr-Glyc | 0.1 | 0.2 | 0.3 | 0.2 |
| 2.5% w/v glycerol, 10 | | | | | |
| mM threonine | | | | | |
| 10 mM glutamic acid, | Glu-medThr-Glyc | 0.1 | 0.2 | 0.2 | 0.1 |
| 2.0% w/v glycerol, 30 | | | | | |
| mM threonine | | | | | |
| 10 mM glutamic acid, | GluGlyc | 0.1 | 0.2 | 0.3 | 0.2 |
| 2.5% w/v glycerol | | | | | |
| 2.5% w/v glycerol | Glyc | 0.1 | 0.2 | 0.3 | 0.2 |
| 10 mM histidine, 7% | RPF | 0.1 | 0.1 | 0.1 | 0.0 |
| w/v sucrose | | | | | |

All formulations contain 25 mg/mL pembrolizumab, 0.02% w/v polysorbate 80 at pH 5.5

TABLE 9
Percent HMW in Evaluated Formulations (5° C.)

| | | 0 | 1 | 2 | 3 | 4 | 8 |
|---|---|---|---|---|---|---|---|
| Formulation | Label | weeks | week | weeks | weeks | weeks | weeks |
| 10 mM glutamic acid, | Glu-highThr | 0.1 | 0.1 | 0.1 | 0.1 | 0.1 | 0.1 |
| 271 mM threonine | | | | | | | |
| 10 mM glutamic acid, | Glu-lowThr-Glyc | 0.1 | 0.1 | 0.1 | 0.2 | 0.2 | 0.2 |
| 2.5% w/v glycerol, | | | | | | | |
| 10 mM threonine | | | | | | | |
| 10 mM glutamic acid, | Glu-medThr-Glyc | 0.1 | 0.1 | 0.1 | 0.1 | 0.1 | 0.2 |
| 2.0% w/v glycerol, | | | | | | | |
| 30 mM threonine | | | | | | | |
| 10 mM glutamic acid, | GluGlyc | 0.1 | 0.1 | 0.1 | 0.1 | 0.2 | 0.2 |
| 2.5% w/v glycerol | | | | | | | |
| 2.5% w/v glycerol | Glyc | 0.1 | 0.1 | 0.1 | 0.1 | 0.1 | 0.2 |
| 1.6% w/v glycerol, | ProGlyc | 0.1 | 0.1 | 0.1 | 0.1 | 0.1 | 0.1 |
| 100 mM proline | | | | | | | |
| 10 mM histidine, | RPF | 0.1 | 0.1 | 0.1 | 0.1 | 0.1 | 0.1 |
| 7% w/v sucrose, | | | | | | | |
| 0.02% w/v PS80 | | | | | | | |
| 10% w/v trehalose | Tre | 0.1 | 0.1 | 0.1 | 0.1 | 0.1 | 0.1 |

TABLE 10
Percent HMW in Evaluated Formulations (25° C.)

| Formulation | Label | 0 weeks | 2 weeks | 4 weeks |
|---|---|---|---|---|
| 2 mM acetate, 10 mM histidine, 2.5% w/v | AceGluGlyc | 0.1 | 0.3 | NT |
| glycerol | | | | |
| 10 mM glutamic acid, 271 mM threonine | Glu-highThr | 0.1 | 0.1 | 0.1 |
| 10 mM glutamic acid, 10% w/v trehalose | Glu-highTreh | 0.1 | 0.1 | 0.2 |
| 10 mM glutamic acid, 271 mM valine | Glu-highVal | 0.1 | 0.1 | 0.1 |
| 10 mM glutamic acid, 2.5% w/v glycerol, 10 | Glu-lowThr-Glyc | 0.1 | 0.2 | 0.3 |
| mM threonine | | | | |
| 10 mM glutamic acid, 2% w/v glycerol, 1% | Glu-lowTreh-Glyc | 0.1 | 0.2 | 0.3 |
| w/v trehalose | | | | |
| 10 mM glutamic acid, 2.5% w/v glycerol, 5 | Glu-lowVal-Glyc | 0.1 | 0.2 | 0.3 |
| mM valine | | | | |
| 10 mM glutamic acid, 2.0% w/v glycerol, 30 | Glu-medThr-Glyc | 0.1 | 0.2 | 0.2 |
| mM threonine | | | | |
| 10 mM glutamic acid, 2.0% w/v glycerol, 65 | Glu-medVal-Glyc | 0.1 | 0.2 | 0.2 |
| mM valine | | | | |
| 10 mM glutamic acid, 2.5% w/v glycerol | GluGlyc | 0.1 | 0.2 | 0.3 |
| 10 mM glutamic acid, 2.5% w/v glycerol, | GluProGlyc | 0.1 | 0.1 | 0.1 |
| 100 mM proline | | | | |
| 10 mM glutamic acid, 2.0% w/v glycerol, | GluSorGlyc | 0.1 | 0.2 | 0.3 |
| 1% w/v sorbitol | | | | |
| 2.5% w/v glycerol | Glyc | 0.1 | 0.2 | 0.3 |
| 10 mM lactic acid, 271 mM threonine | Lac-highThr | 0.1 | 0.1 | 0.1 |
| 10 mM lactic acid, 2.5% w/v glycerol | LacGlyc | 0.1 | 0.2 | 0.3 |
| 1.6% w/v glycerol, 100 mM proline | ProGlyc | 0.1 | 0.1 | 0.2 |
| 10 mM histidine, 7% w/v sucrose, 0.02% | RPF | 0.1 | 0.1 | 0.1 |
| w/v PS80 | | | | |
| 10% w/v trehalose | Tre | 0.1 | 0.1 | 0.1 |

All formulations contain 25 mg/mL pembrolizumab, 0.02% w/v polysorbate 80 at pH 5.5;
NT = not tested

TABLE 11
Percent HMW in Evaluated Formulations (25° C.)

| Formulation | Label | 0 weeks | 2 weeks | 4 weeks |
|---|---|---|---|---|
| 10 mM glutamic acid, 2.5% w/v glycerol | GluGlyc | 0.1 | 0.2 | 0.3 |
| 2.5% w/v glycerol | Glyc | 0.1 | 0.2 | 0.3 |
| 10 mM lactic acid, 2.5% w/v glycerol | LacGlyc | 0.1 | 0.2 | 0.3 |
| 1.6% w/v glycerol, 100 mM proline | ProGlyc | 0.1 | 0.1 | 0.2 |
| 10 mM histidine, 7% w/v sucrose, 0.02% w/v | RPF | 0.1 | 0.1 | 0.1 |
| PS80 | | | | |
| 10% w/v trehalose | Tre | 0.1 | 0.1 | 0.1 |

All formulations contain 25 mg/mL pembrolizumab, 0.02% w/v polysorbate 80 at pH 5.5;
NT = not tested

TABLE 12
Percent HMW in Evaluated Formulations (25° C.)

| Formulation | Label | 0 weeks | 2 weeks | 4 weeks | Δ % HMW |
|---|---|---|---|---|---|
| 10 mM glutamic acid, 271 mM | Glu-highThr | 0.1 | 0.1 | 0.1 | 0.0 |
| threonine | | | | | |
| 10 mM glutamic acid, 10% w/v | Glu-highTreh | 0.1 | 0.1 | 0.2 | 0.1 |
| trehalose | | | | | |
| 10 mM glutamic acid, 271 mM | Glu-highVal | 0.1 | 0.1 | 0.1 | 0.0 |
| valine | | | | | |
| 10 mM glutamic acid, 2.5% w/v | Glu-lowThr-Glyc | 0.1 | 0.2 | 0.3 | 0.2 |
| glycerol, 10 mM threonine | | | | | |
| 10 mM glutamic acid, 2% w/v | Glu-lowTreh-Glyc | 0.1 | 0.2 | 0.3 | 0.2 |
| glycerol, 1% w/v trehalose | | | | | |
| 10 mM glutamic acid, 2.5% w/v | Glu-lowVal-Glyc | 0.1 | 0.2 | 0.3 | 0.2 |
| glycerol, 5 mM valine | | | | | |
| 10 mM glutamic acid, 2.0% w/v | Glu-medThr-Glyc | 0.1 | 0.2 | 0.2 | 0.1 |
| glycerol, 30 mM threonine | | | | | |
| 10 mM glutamic acid, 2.0% w/v | Glu-medVal-Glyc | 0.1 | 0.2 | 0.2 | 0.1 |
| glycerol, 65 mM valine | | | | | |
| 10 mM glutamic acid, 2.5% w/v | GluGlyc | 0.1 | 0.2 | 0.3 | 0.2 |
| glycerol | | | | | |
| 10 mM glutamic acid, 2.5% w/v | GluProGlyc | 0.1 | 0.1 | 0.1 | 0.0 |
| glycerol, 100 mM proline | | | | | |
| 10 mM glutamic acid, 2.0% w/v | GluSorGlyc | 0.1 | 0.2 | 0.3 | 0.2 |
| glycerol, 1% w/v sorbitol | | | | | |
| 2.5% w/v glycerol | Glyc | 0.1 | 0.2 | 0.3 | 0.2 |
| 10 mM histidine, 7% w/v | RPF | 0.1 | 0.1 | 0.1 | 0.0 |
| sucrose, 0.02% w/v PS80 | | | | | |
| 10% w/v trehalose | Tre | 0.1 | 0.1 | 0.1 | 0.0 |

All formulations contain 25 mg/mL pembrolizumab, 0.02% w/v polysorbate 80 at pH 5.5

### Example 4: Further Assessment of 25 mg/mL and 50 mg/mL Pembrolizumab Formulations

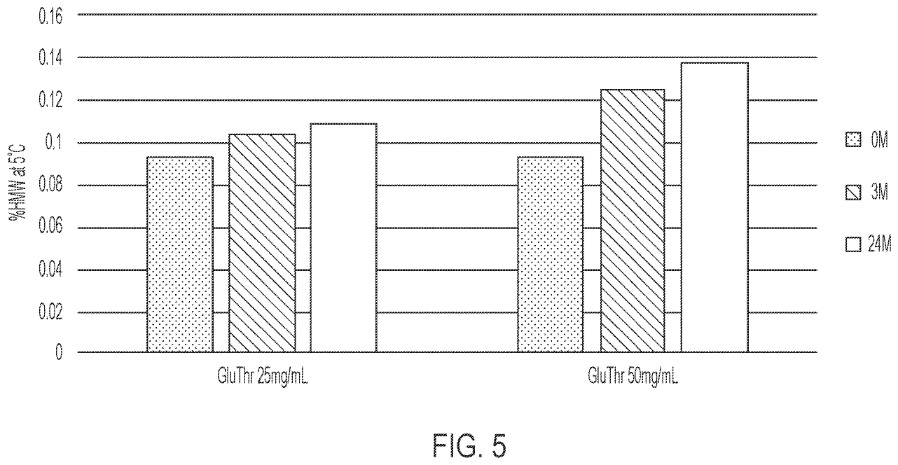

A 50 mg/mL pembrolizumab biosimilar formulation including 250 mM L-threonine, 10 mM L-glutamic acid, 0.02% (w/v) PS80, was compared to a 25 mg/mL pembrolizumab biosimilar formulation containing the same excipients. After 12 weeks, the 50 mg/mL pembrolizumab biosimilar formulation HMW is not significantly higher than HMW species at 25 mg/mL; a similar trend is observed compared to scale-down data. FIG. 5 shows % HMW at 5° C. for 25 mg/mL and 50 mg/mL for 0 months, 3 months, and 24 months (predicted). Formulations containing 25 mg/mL pembrolizumab, 0.02% w/v polysorbate 80 at pH 5.5 were studied for up to 104 weeks (2 years). FIG. 6 shows the results in amount of aggregation in a 104-week stability test at 5° C. (earlier time points discussed in Example 2). The amount of aggregation, shown as percent high molecular weight, or % HMW, over time at 5° C. was measured by size-exclusion chromatography. Glu/Thr (10 mM glutamic acid, 250 mM threonine) showed up to 0.5% HMW less aggregation compared to RPF (10 mM histidine, 7% w/v sucrose; 0.2% HMW) after 104 weeks of stability at 5° C. FIG. 7 shows the results in change in aggregation in a 104-week stability test at 5° C. The change in aggregation, shown as percent high molecular weight, or % HMW, over time at 5° C. was measured by size-exclusion chromatography. Glu/Thr (10 mM glutamic acid, 250 mM threonine, 0.1% HMW) showed less of a change in aggregation compared to RPF (0.2% HMW) after 104 weeks of stability at 5° C. FIG. 8 shows the results in amount of aggregation in a 24-week stability test at 25° C. The amount of aggregation, shown as percent high molecular weight, or % HMW, over time at 25° C. was measured by size-exclusion chromatography. Glu/Thr (10 mM glutamic acid, 250 mM threonine) showed up to 0.4% HMW less aggregation compared to RPF (0.2% HMW) after 24 weeks of stability at 25° C. FIG. 9 shows the results in change in aggregation in a 24-week stability test at 25° C. The change in aggregation, shown as percent high molecular weight, or % HMW, over time at 25° C. was measured by size-exclusion chromatography. Glu/Thr (10 mM glutamic acid, 250 mM threonine; 0.1% HMW) showed less of a change in aggregation compared to RPF (0.2% HMW) after 24 weeks of stability at 25° C.

The results with varying amounts of excipients were also observed. Formulations containing 25 mg/mL pembrolizumab, 0.02% w/v polysorbate 80 at pH 5.5 were studied. FIG. 10 shows the results in amount of aggregation in a 4-week stability test at 25° C. The amount of aggregation, shown as percent high molecular weight, or % HMW, in various buffer-stabilizer combinations was measured by size-exclusion chromatography. Glu-highThr (10 mM glutamic acid, 271 mM threonine) maintained a similar % HMW level to RPF (10 mM histidine, 7% w/v sucrose) over 4 weeks at 25° C. Glu-highThr maintained a lower % HMW than formulations with lower threonine (Glu-lowThr-Glyc, Glu-medThr-Glyc) or no threonine (GluGlyc, Glyc). FIG. 11 shows the results in amount of aggregation in an 8-week stability test at 5° C. The amount of aggregation, shown as percent high molecular weight, or % HMW, in various buffer-stabilizer combinations was measured by size-exclusion chromatography. Glu-highThr (10 mM glutamic acid, 271 mM threonine) maintained a similar % HMW level to RPF (over 8 weeks at 5° C. Glu-highThr maintained a lower % HMW than formulations with lower threonine (Glu-lowThr-Glyc, Glu-medThr-Glyc) or no threonine (GluGlyc, Glyc). FIG. 12 shows the amount of aggregation, shown as percent high molecular weight, or % HMW, over 4 weeks at 25° C. as measured by size-exclusion chromatography. FIG. 13 shows the amount of aggregation, shown as percent high molecular weight, % HMW, in various buffer-stabilizer combinations over 4 weeks at 25° C. as measured by size-exclusion chromatography. FIG. 14 shows the amount of aggregation, shown as percent high molecular weight, % HMW in various buffer-stabilizer combinations over 4 weeks at 25° C. as measured by size-exclusion chromatography.

### Embodiments

The following embodiments are within the scope of the present disclosure. Furthermore, the disclosure encompasses all variations, combinations, and permutations of these embodiments in which one or more limitations, elements, clauses, and descriptive terms from one or more the listed embodiments is introduced into another listed embodiment in this section. For example, any listed embodiment that is dependent on another embodiment can be modified to include one or more limitations found in any other listed embodiment in this section that is dependent on the same base embodiment. Where elements are presented as lists, e.g., in Markush group format, each subgroup of the elements is also disclosed, and any element(s) can be removed from the group. It should be understood that, in general, where the disclosure, or aspects of the disclosure, is/are referred to as comprising particular elements and/or features, certain embodiments of the invention or aspects of the invention consist, or consist essentially of, such elements and/or features. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

A1. A pharmaceutical formulation comprising: (i) about 25 mg/mL to about 200 mg/mL of pembrolizumab or a biosimilar thereof, (ii) a surfactant, and (iii) one or more stabilizing agents, wherein the formulation is liquid with about pH 5.2 to 5.8, and is stable at 2-8° C. for at least 18 months in liquid state, and further wherein the formulation does not comprise histidine and/or a disaccharide.

A2. The pharmaceutical formulation of embodiment A1, wherein the one or more stabilizing agents is at least one amino acid stabilizing agent.

A3. The pharmaceutical formulation of either embodiment A1 or embodiment A2, wherein the one or more stabilizing agents is at least two amino acid stabilizing agents.

A4. The pharmaceutical formulation of embodiment A3, wherein the at least two amino acid stabilizing agents are selected from the group consisting of L-glutamic acid, valine, L-threonine and asparagine.

A5. The pharmaceutical formulation of any one of embodiments A1-A3, wherein the one or more stabilizing agents is two amino acid stabilizing agents, the first amino acid stabilizing agent is L-glutamic acid and the second amino acid stabilizing agent is valine, asparagine or L-threonine.

A6. The pharmaceutical formulation of embodiment A1, wherein the one or more stabilizing agents is a polyol stabilizing agent.

A7. The pharmaceutical formulation of embodiment A1, wherein the one or more stabilizing agents comprises about 10 mM L-glutamic acid and about 271 mM valine, such as 10 mM L-glutamic acid and 271 mM valine.

A8. The pharmaceutical formulation of embodiment A1, wherein the one or more stabilizing agents comprises about 10 mM L-glutamic acid, about 65 mM valine, and about 2% w/v glycerol, such as 10 mM L-glutamic acid, 65 mM valine and 2% w/v glycerol.

A9. The pharmaceutical formulation of embodiment A1, wherein the one or more stabilizing agents comprises about 10 mM L-glutamic acid, about 5 mM valine, and about 2.5% w/v glycerol, such as 10 mM L-glutamic acid, 5 mM valine, and 2.5% w/v glycerol.

A10. The pharmaceutical formulation of embodiment A1, wherein the one or more stabilizing agents comprises about 10 mM L-glutamic acid and about 271 mM L-threonine, such as 10 mM L-glutamic acid and 271 mM L-threonine.

A11. The pharmaceutical formulation of embodiment A1, wherein the one or more stabilizing agents comprises about 10 mM L-glutamic acid and about 250 mM L-threonine, such as 10 mM L-glutamic acid and 250 mM L-threonine.

A12. The pharmaceutical formulation of embodiment A1, wherein the one or more stabilizing agents comprises about 10 mM L-glutamic acid, about 10 mM L-threonine, and about 2.5% w/v glycerol, such as 10 mM L-glutamic acid, 10 mM L-threonine, and 2.5% w/v glycerol.

A13. The pharmaceutical formulation of embodiment A1, wherein the one or more stabilizing agents comprises about 10 mM L-glutamic acid, about 100 mM proline, and about 1.6% w/v glycerol, such as 10 mM L-glutamic acid, 100 mM proline, and 1.6% w/v glycerol.

A14. The pharmaceutical formulation of embodiment A1, wherein the one or more stabilizing agents comprises about 100 mM proline and about 1.6% w/v glycerol, such as 100 mM proline, and 1.6% w/v glycerol.

A15. The pharmaceutical formulation of any of embodiments A1-A14, wherein the formulation does not comprise a buffer.

A16. The pharmaceutical formulation of any of embodiments A1-A15, wherein the surfactant is polysorbate, such as polysorbate 80 (PS 80).

A17. The pharmaceutical formulation of any of embodiments A1-A16, wherein the surfactant is about 0.02% w/v polysorbate 80 (PS 80).

A18. The pharmaceutical formulation of any of embodiments A1-A17, wherein the pH is about 5.5, such as 5.5.

A19. A method of treatment comprising administering the pharmaceutical formulation of any prior embodiments A1-A19, to a subject having or at risk of developing a disease or condition.

A20. The method of embodiment A20, further comprising administering to the subject a second therapeutic composition.

A21. The method of either embodiment A19 or A20, wherein the disease or condition is selected from the group consisting of: cancer, such as melanoma, renal cell carcinoma (RCC), non-small-cell lung cancer, bladder cancer, head and neck squamous cell cancer (HNSCC), classical Hodgkin lymphoma (cHL), primary mediastinal large B-cell lymphoma (PMBCL), urothelial carcinoma, microsatellite instability-high or mismatch repair deficient cancer, microsatellite instability-high or mismatch repair deficient colorectal cancer, gastric cancer, esophageal cancer, cervical cancer, hepatocellular carcinoma (HCC), Merkel cell carcinoma, endometrial carcinoma, tumor mutational burden-high (TMB-H) cancer, cutaneous squamous cell carcinoma (cSCC), triple-negative breast cancer (TNBC), anaplastic thyroid cancer, and/or an infectious disease.

A22. The method of any of embodiments A19-A21, wherein the subject is a mammal.

A23. The method of embodiment A22, wherein the mammal is a human.

A24. The pharmaceutical formulation of or use thereof in any one of embodiments A1-A23, wherein the formulation has 0.1% or less total amount high molecular weight (HMW) species in liquid state after being stored at 5° C. for up to 24 weeks as measured by size exclusion chromatography.

A25. The pharmaceutical formulation of or use thereof in any one of embodiments A1-A23, wherein the formulation has 0.2% or less total amount HMW species in liquid state after being stored at 5° C. for up to 104 weeks as measured by size exclusion chromatography.

A26. The pharmaceutical formulation of or use thereof in any one of embodiments A1-A23, wherein the formulation has less than 0.1% change in total amount of HMW species in liquid state at 5° C. at 104 weeks compared to HMW species at zero weeks.

A27. The pharmaceutical formulation of or use thereof in any one of embodiments A1-A23, wherein the formulation has less than 0.07% change in total amount of HMW species in liquid state at 5° C. at 36 weeks compared to HMW species at zero weeks.

A28. The pharmaceutical formulation of or use thereof in any one of embodiments A1-A27, wherein the formulation further comprises an endoglycosidase hydrolase enzyme.

A29. The pharmaceutical formulation of A28, wherein the endoglycosidase hydrolase enzyme is recombinant human hyaluronidase.

B1 A pharmaceutical formulation comprising: (i) about 25 mg/mL or 50 mg/mL of pembrolizumab or biosimilar thereof, (ii) about 10 mM L-glutamic acid; (iii) about 250 mM L-threonine, about 271 mM valine, about 65 mM valine, or about 100 mM asparagine; and (iv) about 0.02% w/v polysorbate 80, wherein the formulation is liquid with about pH 5.2 to about 5.8, and is stable at 2-8° C. in liquid state, wherein the stable formulation does not comprise a strong buffer and/or a disaccharide stabilizing agent and the stable formulation has less than about 0.06% change in total amount of HMW species in liquid state at 2-8° C. up to 6 months as compared to HMW at zero weeks.

B2. The pharmaceutical formulation of embodiment B1, wherein the formulation consists essentially of 25 mg/mL pembrolizumab biosimilar; (ii) about 10 mM L-glutamic acid; (iii) about 250 mM L-threonine, about 271 mM valine, about 65 mM valine or about 100 mM asparagine; and (iv) about 0.02% w/v polysorbate 80, and further wherein the formulation is stable at 2-8° C. for at least 18 months in which the total amount of high molecular species (HMW) species is 0.2% or less.

B3. The pharmaceutical formulation of embodiment B1, wherein the formulation consists essentially of 25 mg/mL pembrolizumab biosimilar; (ii) about 10 mM L-glutamic acid; (iii) about 250 mM L-threonine, about 271 mM valine, about 65 mM valine or about 100 mM asparagine; and (iv) about 0.02% w/v polysorbate 80, and further wherein the formulation is stable at 2-8° C. for at least 2 years in which the total amount of high molecular species (HMW) species is 0.2% or less.

B4. The pharmaceutical formulation of embodiment B1, wherein the formulation consists essentially of 25 mg/mL pembrolizumab biosimilar; (ii) about 10 mM L-glutamic acid; (iii) about 250 mM L-threonine, about 271 mM valine, about 65 mM valine or about 100 mM asparagine; and (iv) about 0.02% w/v polysorbate 80, and further wherein the formulation is stable at 2-8° C. for at least 3 years.

B5. The pharmaceutical formulation of embodiment B1, wherein the formulation consists essentially of 50 mg/mL pembrolizumab biosimilar; (ii) about 10 mM L-glutamic acid; (iii) about 250 mM L-threonine, about 271 mM valine, about 65 mM valine or about 100 mM asparagine; and (iv) about 0.02% w/v polysorbate 80, and further wherein the formulation is stable at 2-8° C. for at least 18 months in which the total amount of high molecular species (HMW) species is 0.2% or less.

B6. The pharmaceutical formulation of embodiment B1, wherein the formulation consists essentially of 50 mg/mL pembrolizumab biosimilar; (ii) about 10 mM L-glutamic acid; (iii) about 250 mM L-threonine, about 271 mM valine, about 65 mM valine or about 100 mM asparagine; and (iv) about 0.02% w/v polysorbate 80, and further wherein the formulation is stable at 2-8° C. for at least 2 years in which the total amount of high molecular species (HMW) species is 0.2% or less.

B7. The pharmaceutical formulation of embodiment B1, wherein the formulation consists essentially of (i) 50 mg/mL pembrolizumab biosimilar; (ii) about 10 mM L-glutamic acid; (iii) about 250 mM L-threonine, about 271 mM valine, about 65 mM valine or about 100 mM asparagine; and (iv) about 0.02% w/v polysorbate 80, and further wherein the formulation is stable at 2-8° C. for at least 3 years, such as 3 years, 4 years or 5 years.

B8. The pharmaceutical formulation of embodiment B1, wherein the formulation consists essentially of (i) 25 mg/mL pembrolizumab biosimilar; (ii) about 10 mM L-glutamic acid; (iii) about 250 mM L-threonine, about 271 mM valine, about 65 mM valine or about 100 mM asparagine; and (iv) about 0.02% w/v polysorbate 80, and further wherein the formulation is stable at 2-8° C. up to 5 years, such as 5 years.

B9. The pharmaceutical formulation of embodiment B1, wherein the formulation consists essentially of (i) 50 mg/mL pembrolizumab biosimilar; (ii) about 10 mM L-glutamic acid; (iii) about 250 mM L-threonine, about 271 mM valine, about 65 mM valine or about 100 mM asparagine; and (iv) about 0.02% w/v polysorbate 80, and further wherein the formulation is stable at 2-8° C. up to 5 years, such as 5 years.

B10. The pharmaceutical formulation of any one of embodiments B1-B9, wherein the formulation does not comprise histidine, sucrose, trehalose, and/or glycerol.

B11. The pharmaceutical formulation of any one of embodiments B1-B9, wherein the formulation does not comprise histidine, or sucrose.

B12. The pharmaceutical formulation of any one of embodiments B1-B9, wherein the formulation does not comprise histidine, sucrose, or trehalose.

B13. The pharmaceutical formulation of any one of embodiments B1-B12, wherein formulation further does not comprise an antioxidant.

B14. The pharmaceutical formulation of any one of embodiments B1-B14, wherein the pH is about 5.5, such as pH 5.5.

B15. A method of treatment comprising administering the stable pharmaceutical formulation of any prior embodiment B1-B14 to a subject having or at risk of developing a disease or condition.

B16. The method of embodiment B15, further comprising administering to the subject a second therapeutic composition.

B17. The method of either embodiment B15 or B16, wherein the disease or condition is selected from the group consisting of: cancer, such as melanoma, renal cell carcinoma (RCC), non-small-cell lung cancer, bladder cancer, head and neck squamous cell cancer (HNSCC), classical Hodgkin lymphoma (cHL), primary mediastinal large B-cell lymphoma (PMBCL), urothelial carcinoma, microsatellite instability-high or mismatch repair deficient cancer, microsatellite instability-high or mismatch repair deficient colorectal cancer, gastric cancer, esophageal cancer, cervical cancer, hepatocellular carcinoma (HCC), Merkel cell carcinoma, endometrial carcinoma, tumor mutational burden-high (TMB-H) cancer, cutaneous squamous cell carcinoma (cSCC), triple-negative breast cancer (TNBC), anaplastic thyroid cancer, and/or an infectious disease.

B18. The method of any one of embodiment B15-B17, wherein the subject is a mammal.

B19. The method of embodiment B18, wherein the mammal is a human.

B20. The pharmaceutical formulation of any one of embodiments B1-B14, wherein the formulation further comprises an endoglycosidase hydrolase enzyme.

B21. The pharmaceutical formulation of B20, wherein the endoglycosidase hydrolase enzyme is recombinant human hyaluronidase.

C1. A pharmaceutical formulation, comprising: pembrolizumab; 5 mM to 25 mM L-glutamic acid; 100 mM to 300 mM L-threonine; and 0.01% to 0.1% w/v polysorbate, wherein the formulation is liquid, pH range is 5.2-5.8 pH, wherein the formulation does not comprise histidine, sucrose or trehalose, and wherein the formulation has less than 0.07% change in total amount of high molecular weight (HMW) species in liquid state at 5° C. at 36 weeks compared to HMW species at zero weeks and/or wherein the formulation has 0.2% or less high molecular weight (HMW) species in liquid state after being stored at 2°-8° C., including 5° C., for 104 weeks.

C2. The pharmaceutical formulation of embodiment C1, wherein the formulation consists essentially of 50 mg/mL pembrolizumab; (ii) about 10 mM L-glutamic acid; (iii) about 250 mM L-threonine; and (iv) 0.02% w/v polysorbate 80, and wherein the formulation has less than 0.07% change in total amount of high molecular weight (HMW) species in liquid state at 5° C. at 36 weeks compared to HMW species at zero weeks and/or wherein the formulation has 0.2% or less high molecular weight (HMW) species in liquid state after being stored at 5° C. for 104 weeks.

C3. The pharmaceutical formulation of any one of embodiments C1-C2, wherein the formulation does not comprise histidine, sucrose, trehalose and/or glycerol.

C4. The pharmaceutical formulation of any one of embodiments C1-C2, wherein the formulation does not comprise histidine or sucrose.

C5. The pharmaceutical formulation of any one of embodiments C1-C2, wherein the formulation does not comprise histidine, sucrose or trehalose.

C6. The pharmaceutical formulation of any one of embodiments C1-C5, wherein formulation further does not comprise an antioxidant.

C7. The pharmaceutical formulation of any one of embodiments C1-C6, wherein the pH is about 5.5.

C8. A method of treatment comprising administering the stable pharmaceutical formulation of any prior embodiment C1-C7 to a subject having or at risk of developing a disease or condition.

C9. The method of embodiment C8, further comprising administering to the subject a second therapeutic composition.

C10. The method of either embodiment C8 or C9, wherein the disease or condition is selected from the group consisting of: cancer, such as melanoma, renal cell carcinoma (RCC), non-small-cell lung cancer, bladder cancer, head and neck squamous cell cancer (HNSCC), classical Hodgkin lymphoma (cHL), primary mediastinal large B-cell lymphoma (PMBCL), urothelial carcinoma, microsatellite instability-high or mismatch repair deficient cancer, microsatellite instability-high or mismatch repair deficient colorectal cancer, gastric cancer, esophageal cancer, cervical cancer, hepatocellular carcinoma (HCC), Merkel cell carcinoma, endometrial carcinoma, tumor mutational burden-high (TMB-H) cancer, cutaneous squamous cell carcinoma (cSCC), triple-negative breast cancer (TNBC), anaplastic thyroid cancer, and/or an infectious disease.

C11. The method of any one of embodiment C8-C10, wherein the subject is a mammal.

C12. The method of embodiment C18, wherein the mammal is a human.

C13. The pharmaceutical formulation of any one of embodiments C1-C14, wherein the formulation further comprises an endoglycosidase hydrolase enzyme.

C14. The pharmaceutical formulation of C13, wherein the endoglycosidase hydrolase enzyme is recombinant human hyaluronidase.

All publications, patents and patent applications cited in this specification are herein incorporated by reference as if each individual publication or patent application were specifically and individually indicated to be incorporated by reference.

### EQUIVALENTS

Although the foregoing embodiments have been described in some detail by way of illustration and example for purposes of clarity of understanding, it will be readily apparent to those of ordinary skill in the art, in light of the teachings of this disclosure, that certain changes and modifications may be made thereto without departing from the spirit or scope of the disclosed embodiments. Those skilled in the art will recognize, or be able to ascertain, using no more than routine experimentation, many equivalents to the specific embodiments of the invention described herein. Such equivalents are intended to be encompassed by the following claim(s).

1. A pharmaceutical formulation, comprising:
pembrolizumab;
5 mM to 25 mM L-glutamic acid;
100 mM to 300 mM L-threonine;
0.01% to 0.1% w/v polysorbate; and
wherein the formulation is liquid, has a pH in the range of 5.2-5.8, and has 0.2% or less high molecular weight (HMW) species in liquid state after being stored at 2° C. to 8° C. for 104 weeks as measured by size exclusion chromatography.

2. A pharmaceutical formulation, comprising pembrolizumab;
10 mM L-glutamic acid;
65 mM or 271 mM valine; and
0.01% to 0.1% w/v polysorbate;
and wherein the formulation is liquid, has a pH in the range of 5.2-5.8, and has 0.6% or less change in total amount of high molecular weight (HMW) species in liquid state after being stored at 2° C. to 8° C. for up to 6 months as compared to HWM at zero weeks, as measured by size exclusion chromatography.

3. The pharmaceutical formulation of claim 1, wherein the concentration of pembrolizumab is 25 to 200 mg/mL.

4. The pharmaceutical formulation of claim 1, wherein the concentration of pembrolizumab is 25 mg/mL.

5. The pharmaceutical formulation of claim 1, wherein the concentration of L-glutamic acid is 10 mM.

6. The pharmaceutical formulation of claim 1, wherein the concentration of threonine is 250 mM or 271 mM.

7. The pharmaceutical formulation of claim 1, wherein the polysorbate is polysorbate 80.

8. The pharmaceutical formulation of claim 1, wherein the amount of polysorbate is 0.02% w/v or 0.07% w/v polysorbate 80.

9. The pharmaceutical formulation of claim 1, wherein the pH is 5.5.

10. The pharmaceutical formulation of claim 1, wherein the formulation does not comprise does not comprise histidine, sucrose or trehalose, or any combination thereof.

11. The pharmaceutical formulation of claim 1, wherein the formulation further comprises glycerol.

12. The pharmaceutical formulation of claim 1, wherein the formulation does not comprise sucrose, sorbitol, glycerol, or trehalose, or any combination thereof.

13. The pharmaceutical formulation of claim 1, wherein the formulation does not comprise an antioxidant.

14. The pharmaceutical formulation of claim 1, wherein the formulation is suitable for intravenous administration.

15. The pharmaceutical formulation of claim 1, wherein the formulation has 0.1% or less change in total amount of HMW species in liquid state after being stored at 2° C. to 8° C. for 104 weeks as compared to HMW species at zero weeks as measured by size exclusion chromatography.

16. The pharmaceutical formulation of claim 1, wherein the formulation has 0.4% or less HMW species in liquid state after being stored at about 25° C. for 24 weeks as measured by size exclusion chromatography.

17. A method of treating cancer in a subject, comprising administering to the subject an effective amount of the pharmaceutical formulation of claim 1.

18. The method of claim 17, wherein the cancer is selected from the group consisting of melanoma, renal cell carcinoma (RCC), non-small-cell lung cancer (NSCLC), bladder cancer, head and neck squamous cell cancer (HNSCC), classical Hodgkin lymphoma (cHL), primary mediastinal large B-cell lymphoma (PMBCL), urothelial carcinoma, microsatellite instability-high or mismatch repair deficient cancer, microsatellite instability-high or mismatch repair deficient colorectal cancer, gastric cancer, esophageal cancer, cervical cancer, hepatocellular carcinoma (HCC), Merkel cell carcinoma, endometrial carcinoma, tumor mutational burden-high (TMB-H) cancer, cutaneous squamous cell carcinoma (cSCC), triple-negative breast cancer (TNBC), and anaplastic thyroid cancer.

19. The method of claim 17, wherein the effective amount comprises a dosage regimen comprising 200 mg pembrolizumab administered every 3 weeks throughout the course of treatment or 400 mg pembrolizumab administered every 6 weeks throughout the course of treatment.

20. The method of claim 17, further comprising administering to the subject a second therapeutic selected from the group consisting of a chemotherapeutic, sorafenib, axitinib, lenvatinib, ipilimumab, and trastuzumab.
